SEQUENCE LISTING (46 sequences)

SEQ ID NO: 1 - protein, 6 aa, Artificial Sequence
  59R1/59R5 Heavy chain CDR1
SSSGMS

SEQ ID NO: 2 - protein, 17 aa, Artificial Sequence
  59R1/59R5 Heavy chain CDR2
VIASSGSNTYYADSVKG

SEQ ID NO: 3 - protein, 6 aa, Artificial Sequence
  59R1 Heavy chain CDR3
GIFFAI

SEQ ID NO: 4 - protein, 6 aa, Artificial Sequence
  59R5 Heavy chain CDR3
SIFYTT

SEQ ID NO: 5 - protein, 6 aa, Artificial Sequence
  Heavy chain CDR3 consensus sequence
XXFXXX

SEQ ID NO: 6 - protein, 6 aa, Artificial Sequence
  Heavy chain CDR3 variant
SIFYPT

SEQ ID NO: 7 - protein, 6 aa, Artificial Sequence
  Heavy chain CDR3 variant
SSFFAS

SEQ ID NO: 8 - protein, 6 aa, Artificial Sequence
  Heavy chain CDR3 variant
SSFYAS

SEQ ID NO: 9 - protein, 6 aa, Artificial Sequence
  Heavy chain CDR3 variant
SSFFAT

SEQ ID NO: 10 - protein, 6 aa, Artificial Sequence
  Heavy chain CDR3 variant
SIFYPS

SEQ ID NO: 11 - protein, 6 aa, Artificial Sequence
  Heavy chain CDR3 variant
SSFFAN

SEQ ID NO: 12 - protein, 12 aa, Artificial Sequence
  59R1/59R5 Light chain CDR1
RASQSVRSNYLA

SEQ ID NO: 13 - protein, 7 aa, Artificial Sequence
  59R1/59R5 Light chain CDR2
GASSRAT

SEQ ID NO: 14 - protein, 8 aa, Artificial Sequence
  59R1/59R5 Light chain CDR3
QQYSNFPI

SEQ ID NO: 15 - protein, 135 aa, Artificial Sequence
  Heavy chain variable region with predicted signal sequence
MKHLWFFLLLVAAPRWVLSQVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARGIFFAIWGQGTLVTVSSA

SEQ ID NO: 16 - protein, 137 aa, Artificial Sequence
  59R5 Heavy chain variable region with predicted signal sequence
MKHLWFFLLLVAAPRWVLSEVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSIFYTTWGQGTLVTVSSAST

SEQ ID NO: 17 - protein, 118 aa, Artificial Sequence
  59R1 Heavy chain variable region
QVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARGIFFAIWGQGTLVTVSSAST

SEQ ID NO: 18 - protein, 116 aa, Artificial Sequence
  59R5 Heavy chain variable region
EVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSIFYTTWGQGTLVTVSSA

SEQ ID NO: 19 - protein, 116 aa, Artificial Sequence
  Variant 59R1 Heavy chain variable region
QVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSIFYPTWGQGTLVTVSSA

SEQ ID NO: 20 - protein, 116 aa, Artificial Sequence
  Variant 59R1 Heavy chain variable region
QVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSSFFASWGQGTLVTVSSA

SEQ ID NO: 21 - protein, 116 aa, Artificial Sequence
  Variant 59R1 Heavy chain variable region
QVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSSFYASWGQGTLVTVSSA

SEQ ID NO: 22 - protein, 116 aa, Artificial Sequence
  Variant 59R1 Heavy chain variable region
QVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSSFFATWGQGTLVTVSSA

SEQ ID NO: 23 - protein, 116 aa, Artificial Sequence
  Variant 59R1 Heavy chain variable region
QVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSIFYPSWGQGTLVTVSSA

SEQ ID NO: 24 - protein, 116 aa, Artificial Sequence
  Variant 59R1 Heavy chain variable region
QVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSSFFANWGQGTLVTVSSA

SEQ ID NO: 25 - protein, 129 aa, Artificial Sequence
  59R1/59R5 Light chain variable region with predicted signal sequence
MVLQTQVFISLLLWISGAYGDIVLTQSPATLSLSPGERATLSCRASQSVRSNYLAWYQQKPGQAPRLLIYGASSRATGVPARFSGSGSGTDFTLTISSLEPEDFAVYYCQQYSNFPITFGQGTKVEIKR

SEQ ID NO: 26 - protein, 109 aa, Artificial Sequence
  59R1/59R5 Light chain variable region
DIVLTQSPATLSLSPGERATLSCRASQSVRSNYLAWYQQKPGQAPRLLIYGASSRATGVPARFSGSGSGTDFTLTISSLEPEDFAVYYCQQYSNFPITFGQGTKVEIKR

SEQ ID NO: 27 - protein, 460 aa, Artificial Sequence
  59R5 Heavy chain with predicted signal sequence
MKHLWFFLLLVAAPRWVLSEVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSIFYTTWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 28 - protein, 441 aa, Artificial Sequence
  59R5 Heavy chain without predicted signal sequence
EVQLVESGGGLVQPGGSLRLSCAASGFTFSSSGMSWVRQAPGKGLEWVSVIASSGSNTYYADSVKGRFTISRDNSKNTLYLQMNSLRAEDTAVYYCARSIFYTTWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 29 - protein, 235 aa, Artificial Sequence
  59R1/59R5 Light chain with predicted signal sequence
MVLQTQVFISLLLWISGAYGDIVLTQSPATLSLSPGERATLSCRASQSVRSNYLAWYQQKPGQAPRLLIYGASSRATGVPARFSGSGSGTDFTLTISSLEPEDFAVYYCQQYSNFPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - protein, 215 aa, Artificial Sequence
  59R1/59R5 Light chain without predicted signal sequence
DIVLTQSPATLSLSPGERATLSCRASQSVRSNYLAWYQQKPGQAPRLLIYGASSRATGVPARFSGSGSGTDFTLTISSLEPEDFAVYYCQQYSNFPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - DNA, 411 bp, Artificial Sequence
  122M23 Heavy chain variable region with signal sequence
atgaaacacctttggttcttcctcctccttgtcgccgctcctagatgggtcctctcccaagtccaattgcagcagtccggagccgaactcgcacggcctggcgcgagcgtgaagatgagctgcaaggcctcggggtacacctttaccaagtacactatccattgggtcgcccaaaagccaggccagggactcgagtggatcgggtacattaacccgtcgaatgattacaccaactataaccagaccttcaaggataaagcgaccctgacggcggacaagtcctcaaccactgcctacatgcaactgatctcgttgacttcggccgacagcaccgtgttctactgtgctcgcggtactacgccgtactcacttgactactggggacagggaacctccgtgactgtgagctca

SEQ ID NO: 32 - DNA, 354 bp, Artificial Sequence
  122M23 Heavy chain variable region without signal sequence
caagtccaattgcagcagtccggagccgaactcgcacggcctggcgcgagcgtgaagatgagctgcaaggcctcggggtacacctttaccaagtacactatccattgggtcgcccaaaagccaggccagggactcgagtggatcgggtacattaacccgtcgaatgattacaccaactataaccagaccttcaaggataaagcgaccctgacggcggacaagtcctcaaccactgcctacatgcaactgatctcgttgacttcggccgacagcaccgtgttctactgtgctcgcggtactacgccgtactcacttgactactggggacagggaacctccgtgactgtgagctca

SEQ ID NO: 33 - protein, 137 aa, Artificial Sequence
  122M23 Heavy chain variable region with predicted signal sequence
MKHLWFFLLLVAAPRWVLSQVQLQQSGAELARPGASVKMSCKASGYTFTKYTIHWVAQKPGQGLEWIGYINPSNDYTNYNQTFKDKATLTADKSSTTAYMQLISLTSADSTVFYCARGTTPYSLDYWGQGTSVTVSS

SEQ ID NO: 34 - protein, 118 aa, Artificial Sequence
  122M23 Heavy chain variable region without predicted signal sequence
QVQLQQSGAELARPGASVKMSCKASGYTFTKYTIHWVAQKPGQGLEWIGYINPSNDYTNYNQTFKDKATLTADKSSTTAYMQLISLTSADSTVFYCARGTTPYSLDYWGQGTSVTVSS

SEQ ID NO: 35 - DNA, 393 bp, Artificial Sequence
  122M23 Light chain variable region with signal sequence
atgaaacacctgtggttcttcctcctgctggtggcagctcccagatgggtcctgtccgatatcgtgctgactcagagcccagcctcgctggccgtgtcgttgggtcagcgggcgactatctcgtgcagggcctcagaatcagtggacaactacgggatgagcttcatgaactggtttcaacagaagccgggtcaaagccccaaattgcttatctacgctgccagcaaccaaggatccggcgtcccggcacgcttttcgggaagcggctcgggcaccaatttctccctgaatatccaccctatggaagaagatgataccgccgtgtacttctgcctccagtcgaaggaggtgccgtacacgttcggaggaggaactaagctggagattaagcgt

SEQ ID NO: 36 - DNA, 336 bp, Artificial Sequence
  122M23 Light chain variable region without signal sequence
gatatcgtgctgactcagagcccagcctcgctggccgtgtcgttgggtcagcgggcgactatctcgtgcagggcctcagaatcagtggacaactacgggatgagcttcatgaactggtttcaacagaagccgggtcaaagccccaaattgcttatctacgctgccagcaaccaaggatccggcgtcccggcacgcttttcgggaagcggctcgggcaccaatttctccctgaatatccaccctatggaagaagatgataccgccgtgtacttctgcctccagtcgaaggaggtgccgtacacgttcggaggaggaactaagctggagattaagcgt

SEQ ID NO: 37 - protein, 131 aa, Artificial Sequence
  122M23 Light chain variable region with predicted signal sequence underlined
MKHLWFFLLLVAAPRWVLSDIVLTQSPASLAVSLGQRATISCRASESVDNYGMSFMNWFQQKPGQSPKLLIYAASNQGSGVPARFSGSGSGTNFSLNIHPMEEDDTAVYFCLQSKEVPYTFGGGTKLEIKR

SEQ ID NO: 38 - protein, 112 aa, Artificial Sequence
  122M23 Light chain variable region without predicted signal sequence
DIVLTQSPASLAVSLGQRATISCRASESVDNYGMSFMNWFQQKPGQSPKLLIYAASNQGSGVPARFSGSGSGTNFSLNIHPMEEDDTAVYFCLQSKEVPYTFGGGTKLEIKR

SEQ ID NO: 39 - protein, 6 aa, Artificial Sequence
  122M23 Heavy chain CDR1
TKYTIH

SEQ ID NO: 40 - protein, 17 aa, Artificial Sequence
  122M23 Heavy chain CDR2
YINPSNDYTNYNQTFKD

SEQ ID NO: 41 - protein, 11 aa, Artificial Sequence
  122M23 Heavy chain CDR3
ARGTTPYSLDY

SEQ ID NO: 42 - protein, 15 aa, Artificial Sequence
  122M23 Light chain CDR1
RASESVDNYGMSFMN

SEQ ID NO: 43 - protein, 7 aa, Artificial Sequence
  122M23 Light chain CDR2
AASNQGS

SEQ ID NO: 44 - protein, 7 aa, Artificial Sequence
  122M23 Heavy chain CDR3
LQSKEVP

SEQ ID NO: 45 - protein, 2321 aa, Artificial Sequence
  Human NOTCH3
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPAAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA

SEQ ID NO: 46 - protein, 8 aa, Artificial Sequence
  FLAG tag
DYKDDDDK

(19) United States
(12) Patent Application Publication
(10) Pub. No.: US 2017/0023576 A1
(43) Pub. Date: Jan. 26, 2017

(54) NOTCH3 ANTIBODIES AND USES THEREOF

(71) Applicant: ONCOMED PHARMACEUTICALS, INC., Redwood City, CA (US)

(72) Inventor: Belinda CANCILLA, Redwood City, CA (US)

(73) Assignee: ONCOMED PHARMACEUTICALS, INC., Redwood City, CA (US)

(21) Appl. No.: 15/301,161

(22) PCT Filed: Apr. 3, 2015
(86) PCT No.: PCT/US15/24291

§ 371 Date: Sep. 30, 2016

Related U.S. Application Data
(60) Provisional application No. 61/975,620, filed on Apr. 4, 2014.

(51) Int. Cl. G01N 33/574; C07K 16/32; G01N 33/58; A61K 31/7068; C07K 16/30; C07K 16/28; A61K 39/395

(52) U.S. Cl. CPC G01N 33/57438; C07K 16/28; C07K 16/32; A61K 39/39558; A61K 31/7068; C07K 16/303; G01N 33/58; C07K 2317/565; C07K 2317/21; A61K 2039/505

(57) ABSTRACT

Novel antibodies that specifically bind human NOTCH3 are provided. Methods and kits using these antibodies to determine the level of NOTCH3 expression in tumor samples are provided.

### CROSS-REFERENCE TO RELATED APPLICATIONS

This application claims priority benefit of U.S. Provisional Application No. 61/975,620, filed Apr. 4, 2014, which is hereby incorporated by reference herein in its entirety.

### FIELD OF THE INVENTION

The field of this invention generally relates to novel NOTCH3 antibodies and methods of using those antibodies. The invention also provides methods for identifying a tumor, selecting a tumor, or identifying/selecting a patient with a tumor that is likely to be responsive to a Wnt pathway inhibitor, based upon the expression of NOTCH3 in the tumor.

### BACKGROUND OF THE INVENTION

The NOTCH signaling pathway is a universally conserved signal transduction system. It is involved in cell fate determination during development including embryonic pattern formation and post-embryonic tissue maintenance. In addition, NOTCH signaling has been identified as a critical factor in the maintenance of hematopoietic stem cells.

The mammalian NOTCH receptor family includes four members, NOTCH1, NOTCH2, NOTCH3 and NOTCH4. NOTCH receptors are large single-pass type I transmembrane proteins with several conserved structural motifs. The extracellular domain contains a variable number of epidermal growth factor (EGF)-like repeats involved in ligand binding and three cysteine-rich LIN-12/Notch repeats (LNRs) involved in NOTCH heterodimerization. The intracellular domain contains a RAM23 motif involved in binding NOTCH downstream signaling proteins, 7 cdc10/ankyrin repeats also involved in mediating downstream signaling and a PEST domain involved in NOTCH protein degradation.

The extracellular domain of a NOTCH receptor interacts with the extracellular domain of a NOTCH ligand, typically on adjacent cells, resulting in two proteolytic cleavages of the NOTCH receptor. One extracellular cleavage is mediated by an ADAM (A Disintegrin And Metallopeptidase) protease and a second cleavage within the transmembrane domain is mediated by the gamma-secretase complex. This latter cleavage generates the NOTCH intracellular domain (ICD), which translocates to the nucleus where it activates the CBF1, Suppressor of Hairless, Lag-2 (CSL) family of transcription factors as the major downstream effectors to increase transcription of nuclear basic helix-loop-helix transcription factors of the Hairy/Enhancer of Split (HES) family. (See, e.g., Artavanis et al., 1999, Science, 284:770; Brennan and Brown, 2003, Breast Cancer Res., 5:69; Iso et al., 2003, Arterioscler. Thromb. Vasc. Biol., 23:543).

The NOTCH pathway has been linked to the pathogenesis of both hematologic and solid tumors and cancers. Numerous cellular functions and microenvironmental cues associated with tumorigenesis have been shown to be modulated by NOTCH pathway signaling, including cell proliferation, apoptosis, adhesion, and angiogenesis (Leong et al., 2006, Blood, 107:2223-2233). In addition, NOTCH receptors and/or NOTCH ligands have been shown to play potential oncogenic roles in a number of human cancers, including acute myelogenous leukemia, B-cell chronic lymphocytic leukemia, Hodgkin lymphoma, multiple myeloma, T-cell acute lymphoblastic leukemia, brain cancer, breast cancer, cervical cancer, colon cancer, lung cancer, pancreatic cancer, prostate cancer and skin cancer (Leong et al., 2006, Blood, 107:2223-2233).

Pancreatic cancer is the fourth leading cause of cancer deaths in the United States. Patients have a median survival of 6 months and a 5-year survival rate of only 3-5% and this figure has remained relatively unchanged over the past 25 years. Even for patients diagnosed with local disease, the 5-year survival rate is only 15%. The lethal nature of pancreatic cancer stems from its propensity to rapidly disseminate and/or metastasize to the lymphatic system and distant organs. The presence of clinical metastases at the time of diagnosis together with the lack of effective chemotherapies contributes to the high mortality in patients with pancreatic cancer (Iovanna et al., Front. Oncol. 2012; 2:6).

Gemcitabine is the chemotherapeutic drug most commonly used to treat pancreatic cancer and sometimes the gemcitabine is combined with albumin-bound paclitaxel (ABRAXANE). Recently, a combination chemotherapy regimen combining 5-FU, irinotecan, oxaliplatin, and leucovorin (FOLFIRINOX) was shown to nearly double overall survival compared to gemcitabine in patients with metastatic pancreatic cancer. However, the increase in survival was tied to increased toxicity, limiting FOLFIRINOX's use to patients who have a good performance status. In addition, overall survival was still less than 12 months (Conroy et al., N. Engl. J. Med. 2011, 364:1817-25).

As drug discovery and development advances, especially in the cancer field, the “one drug fits all” approach is shifting to a “personalized medicine” strategy. Personalized medicine strategies may include treatment regimens that are based upon cancer biomarkers, including prognostic markers, pharmacodynamic markers, and predictive markers. In general, predictive biomarkers assess the likelihood that a tumor or cancer will be responsive to or sensitive to a specific therapeutic agent, and may allow for the identification and/or the selection of patients most likely to benefit from the use of that agent.

Therefore, there is a need for designing new and targeted therapeutic strategies that can overcome the relative ineffectiveness of current therapies for treatment of pancreatic cancer. Furthermore, there is a clear need to develop assays that are capable of predicting the responsiveness of a tumor/cancer to a particular agent which should allow better patient selection strategies and better therapeutic efficacy.

### SUMMARY OF THE INVENTION

The invention provides novel NOTCH3 antibodies and methods of using those antibodies. The invention also provides methods for identifying a tumor, selecting a tumor, or identifying/selecting a patient with a tumor that is likely to be responsive to a Wnt pathway inhibitor, based upon the expression of NOTCH3 in the tumor.

In one aspect, the invention provides an antibody that specifically binds human NOTCH3. In some embodiments, the antibody specifically binds the extracellular domain of human NOTCH3. In some embodiments, the antibody specifically binds within EGF repeats 9-14 of NOTCH3. In some embodiments, the antibody binds within amino acids 350-580 of NOTCH3 (SEQ ID NO:45).

In some embodiments, the antibody that specifically binds human NOTCH3 comprises: (a) a heavy chain CDR1 comprising TKYTIH (SEQ ID NO:39), a heavy chain CDR2 comprising YINPSNDYTNYNQTFKD (SEQ ID NO:40), and a heavy chain CDR3 comprising ARGTTPYSLDY (SEQ ID NO:41); and (b) a light chain CDR1 comprising RASESVDNYGMSFMN (SEQ ID NO:42), a light chain CDR2 comprising AASNQGS (SEQ ID NO:43), and a light chain CDR3 comprising LQSKEVP (SEQ ID NO:44).

In some embodiments, the antibody that specifically binds NOTCH3 comprises: (a) a heavy chain variable region having at least 90% sequence identity to SEQ ID NO:34; and (b) a light chain variable region having at least 90% sequence identity to SEQ ID NO:38. In some embodiments, the antibody comprises: (a) a heavy chain variable region having at least 95% sequence identity to SEQ ID NO:34; and (b) a light chain variable region having at least 95% sequence identity to SEQ ID NO:38. In other embodiments, the antibody comprises a heavy chain variable region comprising SEQ ID NO:34 and a light chain variable region comprising SEQ ID NO:38.

In some embodiments, the antibody that specifically binds NOTCH3 is a recombinant antibody, a monoclonal antibody, a chimeric antibody, a bispecific antibody, a humanized antibody, a human antibody, an IgG1 antibody, an IgG2 antibody, or an antibody fragment comprising an antigen binding site.

In some embodiments, the antibody that specifically binds NOTCH3 comprises a heavy chain variable region encoded by the plasmid deposited with ATCC as PTA-121156 and a light chain variable region encoded by the plasmid deposited with ATCC as PTA-121155. In some embodiments, the antibody comprises a heavy chain encoded by the plasmid deposited with ATCC as PTA-121156 and a light chain encoded by the plasmid deposited with ATCC as PTA-121155. In some embodiments, the antibody that specifically binds NOTCH3 is produced by a hybridoma deposited with ATCC as PTA-121154.

In another aspect, the invention provides polynucleotides encoding the anti-NOTCH3 antibodies described herein. In some embodiments, the polynucleotides comprise SEQ ID NO:31, SEQ ID NO:32, SEQ ID NO:35, or SEQ ID NO:36. The invention also provides vectors comprising the polynucleotides. The invention also provides cells comprising or producing the polynucleotides and/or vectors described herein. Cells producing the antibodies described herein are also provided, including a hybridoma producing antibody 122M23 deposited with ATCC as deposit no. PTA-121154.

In another aspect, the invention provides methods for using the anti-NOTCH3 antibody. In some embodiments, the anti-NOTCH3 antibody is used to detect NOTCH3 in a tumor sample. In some embodiments, the anti-NOTCH3 antibody is used to detect the extracellular domain of human NOTCH in a pancreatic tumor sample. In some embodiments, the tumor sample is a biopsy sample. In some embodiments, the tumor sample is a formalin-fixed paraffin embedded (FFPE) sample. In some embodiments, the tumor sample is a fresh frozen (FF) sample.

In some embodiments, the tumor sample is a lung tumor, a small-cell lung tumor, a non-small cell lung tumor, a hepatocellular tumor, a gastrointestinal tumor, a pancreatic tumor, a glioblastoma, a cervical cancer tumor, an ovarian tumor, a liver tumor, a bladder tumor, hepatoma, a breast tumor, a colon tumor, a colorectal tumor, an endometrial or uterine tumor, a salivary gland tumor, a kidney tumor, a prostate tumor, a thyroid tumor, or a head and neck tumor.

In some embodiments, the method comprises contacting a pancreatic tumor sample with an anti-NOTCH3 antibody described herein and determining if the antibody binds the sample. In some embodiments, the method further comprises determining the level of NOTCH3 expression in the sample. In some embodiments, the method further comprises comparing the level of NOTCH3 expression in the sample to a pre-determined level of NOTCH3 expression. In some embodiments, the pre-determined level of NOTCH3 expression is a level of NOTCH3 expression in a reference tumor sample, a reference normal tissue sample, a series of reference tumor samples, or a series of reference normal tissue samples.

In some embodiments, the method used to detect and/or determine NOTCH3 expression in a tumor sample is an immunohistochemistry (IHC) assay. In some embodiments, the method used to detect and/or determine NOTCH3 expression in a tumor sample comprises an H-score evaluation.

In another aspect, methods of identifying a pancreatic tumor likely to be responsive to a first antibody that specifically binds human NOTCH2/3 are provided. In some embodiments, a method comprises determining the level of NOTCH3 expression in a sample obtained from the pancreatic tumor, wherein the level of NOTCH3 expression is determined using a second antibody that specifically binds the extracellular domain of human NOTCH3, and the second antibody comprises a heavy chain CDR1 comprising TKYTIH (SEQ ID NO:39), a heavy chain CDR2 comprising YINPSNDYTNYNQTFKD (SEQ ID NO:40), a heavy chain CDR3 comprising ARGTTPYSLDY (SEQ ID NO:41); a light chain CDR1 comprising RASESVDNYGMSFMN (SEQ ID NO:42), a light chain CDR2 comprising AASNQGS (SEQ ID NO:43), and a light chain CDR3 comprising LQSKEVP (SEQ ID NO:44).

In another aspect, methods of identifying a subject with a pancreatic tumor as likely to be responsive to a first antibody that specifically binds human NOTCH2/3 are provided. In some embodiments, a method comprises (a) determining the level of NOTCH3 expression in a sample obtained from the pancreatic tumor, wherein the level of NOTCH3 expression is determined using a second antibody that specifically binds the extracellular domain of human NOTCH3, and the second antibody comprises a heavy chain CDR1 comprising TKYTIH (SEQ ID NO:39), a heavy chain CDR2 comprising YINPSNDYTNYNQTFKD (SEQ ID NO:40), a heavy chain CDR3 comprising ARGTTPYSLDY (SEQ ID NO:41); a light chain CDR1 comprising RASESVDNYGMSFMN (SEQ ID NO:42), a light chain CDR2 comprising AASNQGS (SEQ ID NO:43), and a light chain CDR3 comprising LQSKEVP (SEQ ID NO:44); and (b) identifying the subject as likely to be responsive or non-responsive to treatment with the first antibody that specifically binds human NOTCH2/3 based upon the level of NOTCH3 expression in the pancreatic tumor.

In another aspect, methods for selecting a subject with a pancreatic tumor for treatment with a first antibody that specifically binds human NOTCH2/3 are provided. In some embodiments, a method comprises (a) determining the level of NOTCH3 expression in a sample obtained from the pancreatic tumor, wherein the level of NOTCH3 expression is determined using a second antibody that specifically binds the extracellular domain of human NOTCH3, and the second antibody comprises a heavy chain CDR1 comprising TKYTIH (SEQ ID NO:39), a heavy chain CDR2 comprising YINPSNDYTNYNQTFKD (SEQ ID NO:40), a heavy chain CDR3 comprising ARGTTPYSLDY (SEQ ID NO:41); a light chain CDR1 comprising RASESVDNYGMSFMN (SEQ ID NO:42), a light chain CDR2 comprising AASNQGS (SEQ ID NO:43), and a light chain CDR3 comprising LQSKEVP (SEQ ID NO:44); (b) identifying the pancreatic tumor as likely to be responsive or non-responsive to treatment with the first antibody that specifically binds human NOTCH2/3 based upon the level of NOTCH3 expression in the pancreatic tumor; and (c) selecting the subject for treatment with the first antibody if the pancreatic tumor is identified as likely to be responsive to treatment.

In another aspect, methods for stratifying a pancreatic cancer patient population for treatment with a first antibody that specifically binds human NOTCH2/3 are provided. In some embodiments, a method comprises: (a) determining the level of NOTCH3 expression samples from the patients, wherein the level of NOTCH3 expression is determined using a second antibody that specifically binds the extracellular domain of human NOTCH3, and the second antibody comprises a heavy chain CDR1 comprising TKYTIH (SEQ ID NO:39), a heavy chain CDR2 comprising YINPSNDYTNYNQTFKD (SEQ ID NO:40), a heavy chain CDR3 comprising ARGTTPYSLDY (SEQ ID NO:41); a light chain CDR1 comprising RASESVDNYGMSFMN (SEQ ID NO:42), a light chain CDR2 comprising AASNQGS (SEQ ID NO:43), and a light chain CDR3 comprising LQSKEVP (SEQ ID NO:44); (b) stratifying the patient population based on the expression level of NOTCH3 in the samples.

In some embodiments of the methods described herein, the sample is a biopsy sample. In some embodiments of the methods described herein, the sample is a formalin-fixed paraffin embedded (FFPE) sample. In some embodiments of the methods described herein, the sample is a fresh frozen (FF) sample.

In some embodiments of the methods described herein, the level of NOTCH3 expression in the sample is compared to a pre-determined level of NOTCH3 expression. In some embodiments of the methods described herein, the pre-determined level of NOTCH3 expression is a level of NOTCH3 expression in a reference tumor sample, a reference normal tissue sample, a series of reference tumor samples, or a series of reference normal tissue samples.

In some embodiments of the methods described herein, the level of NOTCH3 expression is determined using an immunohistochemistry (IHC) assay. In some embodiments of the methods described herein, the level of NOTCH3 expression is determined using an assay which comprises an H-score evaluation.

In some embodiments of the methods described herein, the first antibody that specifically binds human NOTCH2/3 comprises: (a) a heavy chain CDR1 comprising SSSGMS (SEQ ID NO:1), a heavy chain CDR2 comprising VIASSGSNTYYADSVKG (SEQ ID NO:2), and a heavy chain CDR3 comprising SIFYTT (SEQ ID NO:4); and (b) a light chain CDR1 comprising RASQSVRSNYLA (SEQ ID NO:12), a light chain CDR2 comprising GASSRAT (SEQ ID NO:13), and a light chain CDR3 comprising QQYSNFPI (SEQ ID NO:14). In some embodiments, the first antibody that specifically binds human NOTCH2/3 comprises: (a) a heavy chain variable region having at least 90% sequence identity to SEQ ID NO:18; and (b) a light chain variable region having at least 90% sequence identity to SEQ ID NO:26. In some embodiments, the first antibody that specifically binds human NOTCH2/3 comprises: (a) a heavy chain variable region having at least 95% sequence identity to SEQ ID NO:18; and (b) a light chain variable region having at least 95% sequence identity to SEQ ID NO:26. In some embodiments, the first antibody that specifically binds human NOTCH2/3 comprises: (a) a heavy chain variable region comprising SEQ ID NO:18; and (b) a light chain variable region comprising SEQ ID NO:26. In some embodiments, the first antibody that specifically binds human NOTCH2/3 comprises the same heavy chain variable region and the same light chain variable region as an antibody encoded by the plasmid deposited with ATCC as PTA-9547. In some embodiments, the first antibody that specifically binds human NOTCH2/3 is encoded by the plasmid deposited with ATCC as PTA-9547.

In another aspect, the invention provides a kit comprising an anti-NOTCH3 antibody described herein. In some embodiments, the kit comprises a diagnostic composition comprising an anti-NOTCH3 antibody described herein.

### BRIEF DESCRIPTIONS OF THE DRAWINGS

FIG. 1A. Activity of OMP-59R5 as a single agent, or in combination with a chemotherapeutic agent in OMP-PN8 pancreatic tumor cells.

FIG. 1B. Activity of OMP-59R5 as a single agent, or in combination with a chemotherapeutic agent in OMP-PN811 pancreatic tumor cells.

FIG. 2. Correlation of NOTCH3 expression and pancreatic tumor growth inhibition with OMP-59R5 in combination with gemcitabine. Extent of pancreatic tumor inhibition by the OMP-59R5 antibody, in combination with gemcitabine, significantly correlates with the levels of NOTCH3 expression in the pancreatic tumor cells.

FIG. 3. Distribution of NOTCH3 expression in pancreatic tumors that are responsive (R) and non-responsive (NR) to OMP-59R5 antibody treatment in combination with gemcitabine. NOTCH3 expression distribution is shown as a boxplot depicting the sample minimum, lower quartile, median, upper quartile and sample maximum.

FIG. 4. NOTCH3 expression in representative pancreatic cancer tissues as assayed by immunohistochemistry (IHC) assay. FIG. 4A shows a pancreatic tumor with high NOTCH3 expression and a pancreatic tumor with moderate NOTCH3 expression. FIG. 4B shows a pancreatic tumor with low NOTCH3 expression and a pancreatic tumor with no NOTCH3 expression. FIG. 4C shows a lung tumor with high NOTCH3 expression (OMPLU90), a lung tumor with low NOTCH3 expression (OMP-LU40), and a lung tumor with no NOTCH3 expression (OMP-LU66).

FIG. 5. Distribution of NOTCH3 expression in pancreatic tumors that are responsive (R) and non-responsive (NR) to OMP-59R5 antibody treatment in combination with gemcitabine as assayed in an IHC assay. NOTCH3 expression distribution is shown as a boxplot depicting the sample minimum, lower quartile, median, upper quartile and sample maximum.

### DETAILED DESCRIPTION OF THE INVENTION

### I. Definitions

To facilitate an understanding of the present invention, a number of terms and phrases are defined below.

The term “biomarker” as used herein may include but is not limited to, nucleic acids and proteins, and variants and fragments thereof. A biomarker may include DNA comprising the entire or partial nucleic acid sequence encoding the biomarker, or the complement of such a sequence. Biomarker nucleic acids useful in the invention are considered to include both DNA and RNA comprising the entire or partial sequence of any of the nucleic acid sequences of interest. Biomarker proteins are considered to comprise the entire or partial amino acid sequence of any of the biomarker proteins or polypeptides.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen-binding site within the variable region of the immunoglobulin molecule. As used herein, the term encompasses intact polyclonal antibodies, intact monoclonal antibodies, single chain antibodies, antibody fragments (such as Fab, Fab′, F(ab′)₂, and Fv fragments), single chain Fv (scFv) antibodies, multispecific antibodies such as bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1, and IgA2), based on the identity of their heavy chain constant domains referred to as alpha, delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and refers to the antigenic determining variable regions of an intact antibody. Examples of antibody fragments include, but are not limited to, Fab, Fab′, F(ab′)₂, and Fv fragments, linear antibodies, single chain antibodies, and multispecific antibodies formed from antibody fragments. “Antibody fragment” as used herein comprises an antigen-binding site or epitope-binding site.

The term “variable region” of an antibody refers to the variable region of an antibody light chain, or the variable region of an antibody heavy chain, either alone or in combination. The variable region of a heavy or light chain each consist of four framework regions (FR) connected by three complementarity determining regions (CDRs), also known as “hypervariable regions”. The CDRs in each chain are held together in close proximity by the framework regions and, with the CDRs from the other chain, contribute to the formation of the antigen-binding site(s) of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda, Md.), and (2) an approach based on crystallographic studies of antigen-antibody complexes (Al-Lazikani et al., 1997, J. Mol. Biol., 273:927-948). In addition, combinations of these two approaches are sometimes used in the art to determine CDRs.

The term “monoclonal antibody” as used herein refers to a homogeneous antibody population involved in the highly specific recognition and binding of a single antigenic determinant or epitope. This is in contrast to polyclonal antibodies that typically include a mixture of different antibodies directed against a variety of different antigenic determinants The term “monoclonal antibody” encompasses both intact and full-length monoclonal antibodies as well as antibody fragments (e.g., Fab, Fab′, F(ab′)₂, Fv), single chain (scFv) antibodies, fusion proteins comprising an antibody portion, and any other modified immunoglobulin molecule comprising an antigen-binding site. Furthermore, “monoclonal antibody” refers to such antibodies made by any number of techniques, including but not limited to, hybridoma production, phage selection, recombinant expression, and transgenic animals.

The term “humanized antibody” as used herein refers to forms of non-human (e.g., murine) antibodies that are specific immunoglobulin chains, chimeric immunoglobulins, or fragments thereof that contain minimal non-human sequences. In some embodiments, humanized antibodies are human immunoglobulins in which residues of the CDRs are replaced by residues from the CDRs of a non-human species (e.g., mouse, rat, rabbit, or hamster) that have the desired specificity, affinity, and/or binding capability. In some embodiments, the Fv framework region residues of a human immunoglobulin are replaced with the corresponding residues in an antibody from a non-human species that has the desired specificity, affinity, and/or binding capability. The humanized antibody can be further modified by the substitution of additional residues either in the Fv framework region and/or within the replaced non-human residues to refine and optimize antibody specificity, affinity, and/or binding capability. In general, the humanized antibody will comprise substantially all of at least one, and typically two, variable domains containing all or substantially all of the CDRs that correspond to the non-human immunoglobulin whereas all or substantially all of the framework regions are those of a human immunoglobulin consensus sequence. The humanized antibody can also comprise at least a portion of an immunoglobulin constant region or domain (Fc), typically that of a human immunoglobulin. Methods used to generate humanized antibodies are well known in the art.

The term “human antibody” as used herein refers to an antibody produced by a human or an antibody having an amino acid sequence corresponding to an antibody produced by a human. A human antibody may be made using any of the techniques known in the art.

The term “chimeric antibody” as used herein refers to an antibody wherein the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and/or binding capability, while the constant regions correspond to sequences in antibodies derived from another species (usually human).

The phrase “affinity-matured antibody” as used herein refers to an antibody with one or more alterations in one or more CDRs thereof that result in an improvement in the affinity of the antibody for antigen, compared to a parent antibody that does not possess those alterations(s). The definition also includes alterations in non-CDR residues made in conjunction with alterations to CDR residues. Preferred affinity-matured antibodies will have nanomolar or even picomolar affinities for the target antigen. Affinity-matured antibodies may be produced by techniques known in the art. In some embodiments, the affinity-matured antibodies are produced by heavy chain variable region and light chain variable region shuffling, random mutagenesis of CDR and/or framework residues, and/or site-directed mutagenesis CDR and/or framework residues.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5 or 8-10 amino acids in a unique spatial conformation.

The terms “antagonist” and “antagonistic” as used herein refer to any molecule that partially or fully blocks, inhibits, reduces, or neutralizes a biological activity of a target and/or signaling pathway (e.g., the NOTCH pathway). The term “antagonist” is used herein to include any molecule that partially or fully blocks, inhibits, reduces, or neutralizes the activity of a protein. Suitable antagonist molecules specifically include, but are not limited to, antagonist antibodies, antibody fragments, soluble receptors, or fragments of soluble receptors.

The terms “modulation” and “modulate” as used herein refer to a change or an alteration in a biological activity. Modulation includes, but is not limited to, stimulating or inhibiting an activity. Modulation may be an increase or a decrease in activity (e.g., a decrease in pathway signaling), a change in binding characteristics, or any other change in the biological, functional, or immunological properties associated with the activity of a protein, pathway, or other biological point of interest.

The terms “selectively binds” or “specifically binds” mean that a binding agent or an antibody reacts or associates more frequently, more rapidly, with greater duration, with greater affinity, or with some combination of the above to the epitope, protein, or target molecule than with alternative substances, including unrelated or related proteins. In certain embodiments “specifically binds” means, for instance, that an antibody binds a protein with a K_{D} of about 0.1 mM or less, but more usually less than about 1 μM. In certain embodiments, “specifically binds” means that an antibody binds a target at times with a K_{D} of at least about 0.1 μM or less, at other times at least about 0.01 μM or less, and at other times at least about 1 nM or less. Because of the sequence identity between homologous proteins in different species, specific binding can include an antibody that recognizes a protein in more than one species (e.g., human NOTCH and mouse NOTCH). Likewise, because of homology within certain regions of polypeptide sequences of different proteins, specific binding can include an antibody (or other polypeptide or binding agent) that recognizes more than one protein (e.g., human NOTCH2 and human NOTCH3). It is understood that, in certain embodiments, an antibody or binding agent that specifically binds a first target may or may not specifically bind a second target. As such, “specific binding” does not necessarily require (although it can include) exclusive binding, i.e. binding to a single target. Thus, a binding agent may, in certain embodiments, specifically bind more than one target. In certain embodiments, multiple targets may be bound by the same binding site on the agent or antibody. For example, an antibody may, in certain instances, comprise two identical antigen-binding sites, each of which specifically binds the same epitope on two or more proteins. In certain alternative embodiments, an antibody may be bispecific or multispecific and comprise at least two antigen-binding sites with differing specificities. Generally, but not necessarily, reference to binding means specific binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies, in certain embodiments, the polypeptides can occur as single chains or associated chains (e.g., dimers).

The terms “polynucleotide” and “nucleic acid” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

“Conditions of high stringency” may be identified by those that: (1) employ low ionic strength and high temperature for washing, for example 15 mM sodium chloride/1.5 mM sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 in 5×SSC (0.75M NaCl, 75 mM sodium citrate) at 42° C.; or (3) employ during hybridization 50% formamide in 5×SSC, 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 50% formamide, followed by a high-stringency wash consisting of 0.1×SSC containing EDTA at 55° C.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST, ALIGN, Megalign, BestFit, GCG Wisconsin Package, and variations thereof. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 residues, at least about 60-80 residues in length or any integral value therebetween. In some embodiments, identity exists over a longer region than 60-80 residues, such as at least about 80-100 residues, and in some embodiments the sequences are substantially identical over the full length of the sequences being compared, such as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Preferably, conservative substitutions in the sequences of the polypeptides and antibodies of the invention do not abrogate the binding of the polypeptide or antibody containing the amino acid sequence, to the antigen to which the polypeptide or antibody binds. Methods of identifying nucleotide and amino acid conservative substitutions which do not eliminate antigen binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The terms “cancer” and “cancerous” as used herein refer to or describe the physiological condition in mammals in which a population of cells are characterized by unregulated cell growth.

The terms “tumor” and “neoplasm” as used herein refer to any mass of tissue that results from excessive cell growth or proliferation, either benign (non-cancerous) or malignant (cancerous) including pre-cancerous lesions.

The term “metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates (e.g., via the bloodstream or lymph) from the primary site of disease to secondary sites.

The terms “cancer stem cell” and “CSC” and “tumor stem cell” and “tumor initiating cell” are used interchangeably herein and refer to cells from a cancer or tumor that: (1) have extensive proliferative capacity; 2) are capable of asymmetric cell division to generate one or more types of differentiated cell progeny wherein the differentiated cells have reduced and/or limited proliferative or developmental potential; and (3) are capable of symmetric cell divisions for self-renewal or self-maintenance. These properties confer on the cancer stem cells the ability to form or establish a tumor or cancer upon serial transplantation into an immunocompromised host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein refers to the ability of a random sample of cells from the tumor to form palpable tumors upon serial transplantation into immunocompromised hosts (e.g., mice). This definition also includes enriched and/or isolated populations of cancer stem cells that form palpable tumors upon serial transplantation into immunocompromised hosts (e.g., mice).

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The term “pharmaceutically acceptable” refers to a product or compound approved (or approvable) by a regulatory agency of the Federal government or a state government or listed in the U.S. Pharmacopeia or other generally recognized pharmacopeia for use in animals, including humans.

The terms “pharmaceutically acceptable excipient, carrier or adjuvant” or “acceptable pharmaceutical carrier” refer to an excipient, carrier or adjuvant that can be administered to a subject, together with at least one binding agent of the present disclosure, and which does not destroy the activity of the binding agent. The excipient, carrier or adjuvant should be non-toxic when administered with a binding agent in doses sufficient to deliver a therapeutic effect.

The terms “effective amount” or “therapeutically effective amount” or “therapeutic effect” refer to an amount of a binding agent, an antibody, polypeptide, polynucleotide, small organic molecule, or other drug effective to “treat” a disease or disorder in a subject or mammal. In the case of cancer, the therapeutically effective amount of a drug (e.g., an antibody) has a therapeutic effect and as such can reduce the number of cancer cells; decrease tumorigenicity, tumorigenic frequency or tumorigenic capacity; reduce the number or frequency of cancer stem cells; reduce the tumor size; reduce the cancer cell population; inhibit and/or stop cancer cell infiltration into peripheral organs including, for example, the spread of cancer into soft tissue and bone; inhibit and/or stop tumor or cancer cell metastasis; inhibit and/or stop tumor or cancer cell growth; relieve to some extent one or more of the symptoms associated with the cancer; reduce morbidity and mortality; improve quality of life; or a combination of such effects. To the extent the agent, for example an antibody, prevents growth and/or kills existing cancer cells, it can be referred to as cytostatic and/or cytotoxic.

The terms “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus those in need of treatment include those already with the disorder; those prone to have the disorder; and those in whom the disorder is to be prevented. In some embodiments, a subject is successfully “treated” according to the methods of the present invention if the patient shows one or more of the following: a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. NOTCH3 Antibodies

The present invention provides antibodies that specifically bind human NOTCH3, also referred to herein as anti-NOTCH3 antibodies. In some embodiments, the antibodies specifically bind the extracellular domain (ECD) of human NOTCH3. In some embodiments, the antibodies bind within EGF repeats 9-14 of human NOTCH3. In some embodiments, the antibodies bind within amino acids 350-580 of human NOTCH3 (SEQ ID NO:45).

In certain embodiments, an anti-NOTCH3 antibody binds NOTCH3 with a dissociation constant (K_{D}) of about 1 μM or less, about 100 nM or less, about 40 nM or less, about 20 nM or less, about 10 nM or less, about 1 nM or less, or about 0.1 nM or less. In certain embodiments, an anti-NOTCH3 antibody binds NOTCH3 with a dissociation constant (K_{D}) of about 1 μM or less, about 100 nM or less, about 40 nM or less, about 20 nM or less, about 10 nM or less, about 1 nM or less, or about 0.1 nM or less. In some embodiments, an anti-NOTCH3 antibody binds NOTCH3 with a K_{D} of about 20 nM or less. In some embodiments, an anti-NOTCH3 antibody binds NOTCH3 with a K_{D} of about 10 nM or less. In some embodiments, an anti-NOTCH3 antibody binds NOTCH3 with a K_{D} of about 1 nM or less. In some embodiments, an anti-NOTCH3 antibody binds NOTCH3 with a K_{D} of about 0.5 nM or less. In some embodiments, an anti-NOTCH3 antibody binds NOTCH3 with a K_{D} of about 0.1 nM or less. In some embodiments, the anti-NOTCH3 antibody binds both human NOTCH3 and a mouse NOTCH. In some embodiments, the dissociation constant of an anti-NOTCH3 antibody is the dissociation constant determined using a NOTCH3 fusion protein comprising at least a portion of the NOTCH3 protein immobilized on a Biacore chip. In some embodiments, the dissociation constant of the antibody to a NOTCH3 protein is the dissociation constant determined using the binding agent captured by an anti-human IgG antibody on a Biacore chip and a NOTCH3 protein.

In certain embodiments, an anti-NOTCH3 antibody binds NOTCH3 with a half maximal effective concentration (EC₅₀) of about 1 μM or less, about 100 nM or less, about 40 nM or less, about 20 nM or less, about 10 nM or less, about 1 nM or less, or about 0.1 nM or less. In certain embodiments, an anti-NOTCH3 antibody binds NOTCH3 with a half maximal effective concentration (EC₅₀) of about 1 μM or less, about 100 nM or less, about 40 nM or less, about 20 nM or less, about 10 nM or less, about 1 nM or less, or about 0.1 nM or less.

In some embodiments, an anti-NOTCH3 antibody is a recombinant antibody. In some embodiments, an anti-NOTCH3 antibody is a monoclonal antibody. In some embodiments, an anti-NOTCH3 antibody is a chimeric antibody. In some embodiments, an anti-NOTCH3 antibody is a humanized antibody. In some embodiments, an anti-NOTCH3 antibody is a human antibody. In some embodiments, an anti-NOTCH3 antibody is an IgA, IgD, IgE, IgG, or IgM antibody. In certain embodiments, an anti-NOTCH3 antibody is an IgG1 antibody. In certain embodiments, an anti-NOTCH3 antibody is an IgG2 antibody. In certain embodiments, an anti-NOTCH3 antibody is an antibody fragment comprising an antigen-binding site. In some embodiments, an anti-NOTCH3 antibody is a bispecific antibody or a multispecific antibody. In some embodiments, an anti-NOTCH3 antibody is a monovalent antibody. In some embodiments, an anti-NOTCH3 antibody is a monospecific antibody. In some embodiments, an anti-NOTCH3 antibody is a bivalent antibody. In some embodiments, an anti-NOTCH3 antibody is conjugated to a cytotoxic moiety. In some embodiments, an anti-NOTCH3 antibody is isolated. In some embodiments, an anti-NOTCH3 antibody is substantially pure.

The anti-NOTCH3 antibodies of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, immunohistochemistry, Western blot analysis, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well-known in the art (see, e.g., Ausubel et al., Editors, 1994-present, Current Protocols in Molecular Biology, John Wiley & Sons, Inc., New York, N.Y.).

For example, the specific binding of an antibody to human NOTCH3 may be determined using ELISA. An ELISA assay comprises preparing antigen, coating wells of a 96 well microtiter plate with antigen, adding an anti-NOTCH3 antibody conjugated to a detectable compound such as an enzymatic substrate (e.g. horseradish peroxidase or alkaline phosphatase) to the well, incubating for a period of time and detecting the presence of the antibody bound to the antigen. In some embodiments, an anti-NOTCH3 antibody is not conjugated to a detectable compound, but instead a second conjugated antibody that recognizes the anti-NOTCH3 antibody (e.g., an anti-Fc antibody) and is conjugated to a detectable compound is added to the well. In some embodiments, instead of coating the well with the antigen, the anti-NOTCH3 antibody can be coated to the well and a second antibody conjugated to a detectable compound can be added following the addition of the antigen to the coated well. One of skill in the art would be knowledgeable as to the parameters that can be modified to increase the signal detected as well as other variations of ELISAs known in the art.

In another example, the specific binding of an antibody to human NOTCH3 may be determined using FACS. A FACS screening assay may comprise generating a cDNA construct that expresses an antigen as a fusion protein (e.g., NOTCH3 ECD-Fc or NOTCH3 ECD-CD4TM), transfecting the construct into cells, expressing the antigen on the surface of the cells, mixing the anti-NOTCH3 antibody with the transfected cells, and incubating for a period of time. The cells bound by the anti-NOTCH3 antibody may be identified using a secondary antibody conjugated to a detectable compound (e.g., PE-conjugated anti-Fc antibody) and a flow cytometer. One of skill in the art would be knowledgeable as to the parameters that can be modified to optimize the signal detected as well as other variations of FACS that may enhance screening (e.g., screening for blocking antibodies).

The binding affinity of an antibody or other binding-agent to an antigen (e.g., NOTCH3) and the off-rate of an antibody-antigen interaction can be determined by competitive binding assays. One example of a competitive binding assay is a radioimmunoassay comprising the incubation of labeled antigen (e.g., ³H or ¹²⁵I), or fragment or variant thereof, with the antibody of interest in the presence of increasing amounts of unlabeled antigen followed by the detection of the antibody bound to the labeled antigen. The affinity of the antibody for the antigen and the binding off-rates can be determined from the data by Scatchard plot analysis. In some embodiments, Biacore kinetic analysis is used to determine the binding on and off rates of antibodies or agents that bind an antigen (e.g., NOTCH3). In some embodiments, Biacore kinetic analysis comprises analyzing the binding and dissociation of antibodies from chips with immobilized antigen (e.g., NOTCH3) on their surface. In some embodiments, Biacore kinetic analysis comprises analyzing the binding and dissociation of antigen (e.g., NOTCH3) from chips with immobilized antibody (e.g., anti-NOTCH3 antibody) on their surface.

In certain embodiments, the invention provides an anti-NOTCH3 antibody that specifically binds human NOTCH3, wherein the anti-NOTCH3 antibody comprises one, two, three, four, five, and/or six of the CDRs of antibody 122M23 (see Table 1). In some embodiments, the anti-NOTCH3 antibody comprises one or more of the CDRs of 122M23; two or more of the CDRs of 122M23; three or more of the CDRs of 122M23; four or more of the CDRs of 122M23; five or more of the CDRs of 122M23; or all six of the CDRs of 122M23.

TABLE 1

| | 122M23 |
|---|---|
| HC CDR1 | TKYTIH (SEQ ID NO: 39) |
| HC CDR2 | YINPSNDYTNYNQTFKD (SEQ ID NO: 40) |
| HC CDR3 | ARGTTPYSLDY (SEQ ID NO: 41) |
| LC CDR1 | RASESVDNYGMSFMN (SEQ ID NO: 42) |
| LC CDR2 | AASNQGS (SEQ ID NO: 43) |
| LC CDR3 | LQSKEVP (SEQ ID NO: 44) |

In some embodiments, the invention provides an antibody that specifically binds human NOTCH3, wherein the antibody comprises: (a) a heavy chain CDR1 comprising TKYTIH (SEQ ID NO:39), a heavy chain CDR2 comprising YINPSNDYTNYNQTFKD (SEQ ID NO:40), and a heavy chain CDR3 comprising ARGTTPYSLDY (SEQ ID NO:41); and (b) a light chain CDR1 comprising RASESVDNYGMSFMN (SEQ ID NO:42), a light chain CDR2 comprising AASNQGS (SEQ ID NO:43), and a light chain CDR3 comprising LQSKEVP (SEQ ID NO:44).

In certain embodiments, the invention provides an anti-NOTCH3 antibody that specifically binds human NOTCH3, wherein the anti-NOTCH3 antibody comprises: (a) a heavy chain CDR1 comprising TKYTIH (SEQ ID NO:39), or a variant thereof comprising 1, 2, 3, or 4 amino acid substitutions; (b) a heavy chain CDR2 comprising YINPSNDYTNYNQTFKD (SEQ ID NO:40), or a variant thereof comprising 1, 2, 3, or 4 amino acid substitutions; (c) a heavy chain CDR3 comprising ARGTTPYSLDY (SEQ ID NO:41), or a variant thereof comprising 1, 2, 3, or 4 amino acid substitutions; (d) a light chain CDR1 comprising RASESVDNYGMSFMN (SEQ ID NO:42), or a variant thereof comprising 1, 2, 3, or 4 amino acid substitutions; (e) a light chain CDR2 comprising AASNQGS (SEQ ID NO:43), or a variant thereof comprising 1, 2, 3, or 4 amino acid substitutions; and (f) a light chain CDR3 comprising LQSKEVP (SEQ ID NO:44), or a variant thereof comprising 1, 2, 3, or 4 amino acid substitutions. In certain embodiments, the amino acid substitutions are conservative substitutions. In some embodiments, the substitutions are made as part of a germline humanization process.

In certain embodiments, the invention provides an anti-NOTCH3 antibody that specifically binds NOTCH3, wherein the antibody comprises a heavy chain variable region having at least about 80% sequence identity to SEQ ID NO:34 and/or a light chain variable region having at least 80% sequence identity to SEQ ID NO:38. In certain embodiments, the anti-NOTCH3 antibody comprises a heavy chain variable region having at least about 85%, at least about 90%, at least about 95%, at least about 97%, or at least about 99% sequence identity to SEQ ID NO:34. In certain embodiments, the anti-NOTCH3 antibody comprises a light chain variable region having at least about 85%, at least about 90%, at least about 95%, at least about 97%, or at least about 99% sequence identity to SEQ ID NO:38. In certain embodiments, the anti-NOTCH3 antibody comprises a heavy chain variable region having at least 95% sequence identity to SEQ ID NO:34 and a light chain variable region having at least 95% sequence identity to SEQ ID NO:38. In certain embodiments, the anti-NOTCH3 antibody comprises a heavy chain variable region comprising SEQ ID NO:34 and a light chain variable region comprising SEQ ID NO:38. In certain embodiments, the anti-NOTCH3 antibody comprises a heavy chain variable region of SEQ ID NO:34 and a light chain variable region of SEQ ID NO:38.

In certain embodiments, an anti-NOTCH3 antibody comprises a heavy chain comprising SEQ ID NO:28 and a light chain comprising SEQ ID NO:30.

In certain embodiments, an anti-NOTCH3 antibody comprises the heavy chain variable region and light chain variable region of the 122M23 antibody. In certain embodiments, an anti-NOTCH3 antibody comprises the heavy chain and light chain of the 122M23 antibody (with or without the leader sequence). In certain embodiments, an anti-NOTCH3 antibody is the 122M23 antibody. In certain embodiments, an anti-NOTCH3 antibody comprises the heavy chain variable region and/or light chain variable region of the 122M23 antibody in a chimeric form of the antibody. In certain embodiments, an anti-NOTCH3 antibody comprises the heavy chain variable region and/or light chain variable region of the 122M23 antibody in a humanized form of the antibody. In certain embodiments, an anti-NOTCH3 antibody comprises the heavy chain CDRs and/or light chain CDRs of the 122M23 antibody in a humanized form of the antibody. In some embodiments, the humanized version of 122M23 is an IgG1 antibody. In some embodiments, the humanized version of 122M23 is an IgG2 antibody.

In certain embodiments, an anti-NOTCH3 antibody comprises, consists essentially of, or consists of, the antibody 122M23.

In some embodiments, an anti-NOTCH3 antibody comprises a heavy chain variable region encoded by the plasmid deposited with American Type Culture Collection (ATCC), and designated PTA-121156. In some embodiments, an anti-NOTCH3 antibody comprises a light chain variable region encoded by the plasmid deposited with ATCC and designated PTA-121155. In some embodiments, an anti-NOTCH3 antibody comprises a heavy chain variable region encoded by the plasmid deposited with ATCC and designated PTA-121156 and a light chain variable region encoded by the plasmid deposited with ATCC and designated PTA-121155. In some embodiments, an anti-NOTCH3 antibody comprises a heavy chain encoded by the plasmid deposited with ATCC and designated PTA-121156. In some embodiments, an anti-NOTCH3 antibody comprises a light chain encoded by the plasmid deposited with ATCC and designated PTA-121155. In some embodiments, an anti-NOTCH3 antibody comprises a heavy chain encoded by the plasmid deposited with ATCC and designated PTA-121156 and a light chain encoded by the plasmid deposited with ATCC and designated PTA-121155. In some embodiments, an anti-NOTCH3 antibody is produced by the hybridoma deposited with ATCC and designated PTA-121154.

The invention provides polypeptides, including, but not limited to, antibodies that specifically bind human NOTCH3. In certain embodiments, the polypeptide comprises one, two, three, four, five, and/or six of the CDRs of antibody 122M23 (see Table 1 herein). In some embodiments, the polypeptide comprises CDRs with up to four (i.e., 0, 1, 2, 3, or 4) amino acid substitutions per CDR. In certain embodiments, the heavy chain CDR(s) are contained within a heavy chain variable region. In certain embodiments, the light chain CDR(s) are contained within a light chain variable region.

In some embodiments, the invention provides a polypeptide that specifically binds human NOTCH3, wherein the polypeptide comprises an amino acid sequence having at least about 80% sequence identity to SEQ ID NO:33, SEQ ID NO:34, SEQ ID NO:37, or SEQ ID NO:38. In certain embodiments, the polypeptide comprises an amino acid sequence having at least about 85%, at least about 90%, at least about 95%, at least about 97%, or at least about 99% sequence identity to SEQ ID NO:33, SEQ ID NO:34, SEQ ID NO:37, or SEQ ID NO:38. In certain embodiments, the polypeptide comprises an amino acid sequence having at least about 95% sequence identity to SEQ ID NO:33, SEQ ID NO:34, SEQ ID NO:37, or SEQ ID NO:38. In certain embodiments, the polypeptide comprises an amino acid sequence comprising SEQ ID NO:33 and/or an amino acid sequence comprising SEQ ID NO:37. In certain embodiments, the polypeptide comprises an amino acid sequence comprising SEQ ID NO:34 and/or an amino acid sequence comprising SEQ ID NO:38. In certain embodiments, the polypeptide comprises an amino acid sequence comprising SEQ ID NO:33 and an amino acid sequence comprising SEQ ID NO:37. In certain embodiments, the polypeptide comprises an amino acid sequence comprising SEQ ID NO:34 and an amino acid sequence comprising SEQ ID NO:38. In some embodiments, a polypeptide that specifically binds human NOTCH3 comprises a polypeptide comprising a sequence selected from the group consisting of: SEQ ID NO:33, SEQ ID NO:34, SEQ ID NO:37, or SEQ ID NO:38.

Many proteins, including antibodies, contain a signal sequence that directs the transport of the proteins to various locations. Signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell's outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence. Although there is usually one specific cleavage site, more than one cleavage site may be recognized and/or may be used by a signal peptidase resulting in a non-homogenous N-terminus of the polypeptide. For example, the use of different cleavage sites within a signal sequence can result in a polypeptide expressed with different N-terminal amino acids. Accordingly, in some embodiments, the polypeptides as described herein may comprise a mixture of polypeptides with different N-termini. In some embodiments, the N-termini differ in length by 1, 2, 3, 4, or 5 amino acids. In some embodiments, the polypeptide is substantially homogeneous, i.e., the polypeptides have the same N-terminus. In some embodiments, the signal sequence of the polypeptide comprises one or more (e.g., one, two, three, four, five, six, seven, eight, nine, ten, etc.) amino acid substitutions and/or deletions as compared to a “native” or “parental” signal sequence. In some embodiments, the signal sequence of the polypeptide comprises amino acid substitutions and/or deletions that allow one cleavage site to be dominant, thereby resulting in a substantially homogeneous polypeptide with one N-terminus. In some embodiments, a signal sequence of the polypeptide affects the expression level of the polypeptide, e.g., increased expression or decreased expression.

In certain embodiments, an anti-NOTCH3 antibody competes for specific binding to NOTCH3 with an antibody that comprises a heavy chain variable region comprising SEQ ID NO:33 or SEQ ID NO:34, and a light chain variable region comprising SEQ ID NO:37 or SEQ ID NO:38. In certain embodiments, an anti-NOTCH3 antibody competes with antibody 122M23 for specific binding to human NOTCH3. In certain embodiments, an anti-NOTCH3 antibody binds the same epitope, or essentially the same epitope, on NOTCH3 as antibody 122M23.

In some embodiments, the anti-NOTCH3 antibodies are monoclonal antibodies. Monoclonal antibodies can be prepared using hybridoma methods known to one of skill in the art. In some embodiments, using the hybridoma method, a mouse, hamster, or other appropriate host animal, is immunized as described above to elicit from lymphocytes the production of antibodies that specifically bind the immunizing antigen. In some embodiments, lymphocytes can be immunized in vitro. In some embodiments, the immunizing antigen can be a human protein or a portion thereof. In some embodiments, the immunizing antigen can be a mouse protein or a portion thereof.

Following immunization, lymphocytes are isolated and fused with a suitable myeloma cell line using, for example, polyethylene glycol. The hybridoma cells are selected using specialized media as known in the art and unfused lymphocytes and myeloma cells do not survive the selection process. Hybridomas that produce monoclonal antibodies directed specifically against a chosen antigen may be identified by a variety of methods including, but not limited to, immunoprecipitation, immunoblotting, and in vitro binding assays (e.g., flow cytometry, FACS, ELISA, and radioimmunoassay). The hybridomas can be propagated either in in vitro culture using standard methods (J. W. Goding, 1996, Monoclonal Antibodies: Principles and Practice, 3^{rd} Edition, Academic Press, San Diego, Calif.) or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art. The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using standard techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins.

In certain other embodiments, recombinant monoclonal antibodies, or fragments thereof, can be isolated from phage display libraries expressing variable domains or CDRs of a desired species by techniques well known in the art.

The polynucleotide(s) encoding a monoclonal antibody can be modified, for example, by using recombinant DNA technology to generate alternative antibodies. In some embodiments, the constant domains of the light and heavy chains of, for example, a mouse monoclonal antibody can be substituted for those regions of, for example, a human antibody to generate a chimeric antibody, or for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

In some embodiments, a monoclonal antibody against human NOTCH3 is a humanized antibody. Typically, humanized antibodies are human immunoglobulins in which residues from the CDRs are replaced by residues from a CDR of a non-human species (e.g., mouse, rat, rabbit, hamster, etc.) that have the desired specificity, affinity, and/or binding capability using methods known to one skilled in the art. In some embodiments, the Fv framework region residues of a human immunoglobulin are replaced with the corresponding residues in an antibody from a non-human species that has the desired specificity, affinity, and/or binding capability. In some embodiments, a humanized antibody can be further modified by the substitution of additional residues either in the Fv framework region and/or within the replaced non-human residues to refine and optimize antibody specificity, affinity, and/or capability. In general, a humanized antibody will comprise substantially all of at least one, and typically two or three, variable domain regions containing all, or substantially all, of the CDRs that correspond to the non-human immunoglobulin whereas all, or substantially all, of the framework regions are those of a human immunoglobulin consensus sequence. In some embodiments, a humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject. One skilled in the art would be able to obtain a functional humanized antibody with reduced immunogenicity following known techniques.

In certain embodiments, the anti-NOTCH3 antibody is a human antibody. Human antibodies can be directly prepared using various techniques known in the art. In some embodiments, human antibodies may be generated from immortalized human B lymphocytes immunized in vitro or from lymphocytes isolated from an immunized individual. In either case, cells that produce an antibody directed against a target antigen can be generated and isolated. In some embodiments, the human antibody can be selected from a phage library, where that phage library expresses human antibodies. Alternatively, phage display technology can be used to produce human antibodies and antibody fragments in vitro, from immunoglobulin variable domain gene repertoires from unimmunized donors. Once antibodies are identified, affinity maturation strategies known in the art, including but not limited to, chain shuffling and site-directed mutagenesis, may be employed to generate high affinity human antibodies.

In some embodiments, human antibodies can be made in transgenic mice that contain human immunoglobulin loci. Upon immunization these mice are capable of producing the full repertoire of human antibodies in the absence of endogenous immunoglobulin production.

In certain embodiments, the antibodies (or other polypeptides) described herein may be monospecific. In certain embodiments, each of the one or more antigen-binding sites that an antibody contains is capable of binding (or binds) a homologous epitope on NOTCH3. In certain embodiments, an antigen-binding site of a monospecific antibody described herein is capable of binding (or binds), for example, NOTCH3 and NOTCH2 (i.e., the same epitope is found on both NOTCH3 and NOTCH2 proteins).

In certain embodiments, the anti-NOTCH3 antibody is an antibody fragment. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)₂ fragment produced by pepsin digestion of an antibody molecule. In some embodiments, antibody fragments include a Fab fragment generated by reducing the disulfide bridges of an F(ab′)₂ fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries to allow rapid and effective identification of monoclonal Fab fragments with the desired specificity for a NOTCH3 or derivatives, fragments, analogs or homologs thereof. In some embodiments, antibody fragments are linear antibody fragments. In certain embodiments, antibody fragments are monospecific or bispecific.

It can further be desirable, especially in the case of antibody fragments, to modify an antibody in order to alter (e.g., increase or decrease) its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

Heteroconjugate antibodies are also within the scope of the present invention. Heteroconjugate antibodies are composed of two covalently joined antibodies. Such antibodies have, for example, been proposed to target immune cells to unwanted cells. It is also contemplated that the heteroconjugate antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

For the purposes of the present invention, it should be appreciated that modified antibodies can comprise any type of variable region that provides for the association of the antibody with the target (i.e., human NOTCH3). In this regard, the variable region may comprise or be derived from any type of mammal that can be induced to mount a humoral response and generate immunoglobulins against the desired antigen. As such, the variable region of the modified antibodies can be, for example, of human, murine, non-human primate (e.g. cynomolgus monkeys, macaques, etc.) or rabbit origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence modification and/or alteration. Although the CDRs may be derived from an antibody of the same class or even subclass as the antibody from which the framework regions are derived, it is envisaged that the CDRs may be derived from an antibody of different class and often from an antibody from a different species. It may not be necessary to replace all of the CDRs with all of the CDRs from the donor variable region to transfer the antigen binding capacity of one variable domain to another. Rather, it may only be necessary to transfer those residues that are required to maintain the activity of the antigen-binding site.

Alterations to the variable region notwithstanding, those skilled in the art will appreciate that the modified antibodies of this invention will comprise antibodies (e.g., full-length antibodies or immunoreactive fragments thereof) in which at least a fraction of one or more of the constant region domains has been deleted or otherwise altered so as to provide desired biochemical characteristics such as increased tumor localization or increased serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. The modified antibodies disclosed herein may comprise alterations or modifications to one or more of the three heavy chain constant domains (CH1, CH2 or CH3) and/or to the light chain constant domain (CL). In some embodiments, one or more domains are partially or entirely deleted from the constant regions of the modified antibodies. In some embodiments, the modified antibodies will comprise domain deleted constructs or variants wherein the entire CH2 domain has been removed (ΔCH2 constructs). In some embodiments, the omitted constant region domain is replaced by a short amino acid spacer (e.g., 10 amino acid residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete the part of one or more constant region domains that control a specific effector function (e.g. complement C1q binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell cytotoxicity or ADCC), release of inflammatory mediators, placental transfer, and control of immunoglobulin production.

In certain embodiments, the modified antibodies provide for altered effector functions that, in turn, affect the biological profile of the administered antibody. For example, in some embodiments, the deletion or inactivation (through point mutations or other means) of a constant region domain may reduce Fc receptor binding of the circulating modified antibody thereby increasing cancer cell localization and/or tumor penetration. In other embodiments, the constant region modifications increase the serum half-life of the antibody. In other embodiments, the constant region modifications reduce the serum half-life of the antibody. In some embodiments, the constant region is modified to eliminate disulfide linkages or oligosaccharide moieties. Modifications to the constant region in accordance with this invention may easily be made using well known biochemical or molecular engineering techniques.

In certain embodiments, an anti-NOTCH3 antibody is an antibody does not have one or more effector functions. For instance, in some embodiments, the antibody has no ADCC activity, and/or no complement-dependent cytotoxicity (CDC) activity. In certain embodiments, the antibody does not bind an Fc receptor, and/or complement factors. In certain embodiments, the antibody has no effector function.

The present invention further embraces variants and equivalents which are substantially homologous to the chimeric, humanized, and human antibodies, or antibody fragments thereof, set forth herein. These can contain, for example, conservative substitution mutations, i.e. the substitution of one or more amino acids by similar amino acids. For example, conservative substitution refers to the substitution of an amino acid with another amino acid within the same general class such as, for example, one acidic amino acid with another acidic amino acid, one basic amino acid with another basic amino acid or one neutral amino acid by another neutral amino acid. What is intended by a conservative amino acid substitution is well known in the art and described herein.

Thus, the present invention provides methods for producing an antibody that binds NOTCH3, including bispecific antibodies that specifically bind both NOTCH3 and a second target. In some embodiments, the method for producing an antibody that binds NOTCH3 comprises using hybridoma techniques. In some embodiments, a method for producing an antibody that binds human NOTCH3 is provided. In some embodiments, the method comprises EGF repeats 9-14 of human NOTCH3. In some embodiments, the method comprises using amino acids 40-1643 of human NOTCH3 (SEQ ID NO:45). In some embodiments, the method comprises using amino acids 40-580 of human NOTCH3 (SEQ ID NO:45). In some embodiments, the method comprises using amino acids 350-580 of human NOTCH3 (SEQ ID NO:45). In some embodiments, the method of generating an antibody that binds NOTCH3 comprises screening a human phage library. The present invention further provides methods of identifying an antibody that binds NOTCH3. In some embodiments, the antibody is identified by FACS screening for binding to NOTCH3, or a portion thereof. In some embodiments, the antibody is identified by screening using ELISA for binding to NOTCH3, or a portion thereof.

In some embodiments, a method of generating an antibody to human NOTCH3 protein comprises immunizing a mammal with a polypeptide comprising amino acids 40-1643 of human NOTCH3. In some embodiments, a method of generating an antibody to human NOTCH3 protein comprises immunizing a mammal with a polypeptide comprising at least a portion of amino acids 40-1643 of human NOTCH3. In some embodiments, a method of generating an antibody to human NOTCH3 protein comprises immunizing a mammal with a polypeptide comprising amino acids 350-580 of human NOTCH3. In some embodiments, the method further comprises isolating antibodies or antibody-producing cells from the mammal. In some embodiments, a method of generating a monoclonal antibody which binds NOTCH3 protein comprises: (a) immunizing a mammal with a polypeptide comprising at least a portion of amino acids 350-5580 of human NOTCH3; (b) isolating antibody producing cells from the immunized mammal; (c) fusing the antibody-producing cells with cells of a myeloma cell line to form hybridoma cells. In some embodiments, the method further comprises (d) selecting a hybridoma cell expressing an antibody that binds NOTCH3 protein. In certain embodiments, the mammal is a mouse.

In some embodiments, a method of producing an antibody to NOTCH3 comprises identifying an antibody using a membrane-bound heterodimeric molecule comprising a single antigen-binding site. In some non-limiting embodiments, the antibody is identified using methods and polypeptides described in International Publication WO 2011/100566.

In some embodiments, a method of producing an antibody to human NOTCH3 comprises screening an antibody-expressing library for antibodies that bind human NOTCH3. In some embodiments, the antibody-expressing library is a phage library. In some embodiments, the screening comprises panning. In some embodiments, the antibody-expressing library is a phage library. In some embodiments, the antibody-expressing library is a mammalian cell library. In some embodiments, the antibody-expressing library is screened using at least a portion of amino acids 40-1643 of human NOTCH3.

In certain embodiments, the antibodies described herein are isolated. In certain embodiments, the antibodies described herein are substantially pure.

In some embodiments of the present invention, the anti-NOTCH3 antibodies are polypeptides. The polypeptides can be recombinant polypeptides, natural polypeptides, or synthetic polypeptides comprising an antibody, or fragment thereof, that bind NOTCH3. It will be recognized in the art that some amino acid sequences of the invention can be varied without significant effect of the structure or function of the protein. Thus, the invention further includes variations of the polypeptides which show substantial activity or which include regions of an antibody, or fragment thereof, against human NOTCH3. In some embodiments, amino acid sequence variations of NOTCH3-binding polypeptides include deletions, insertions, inversions, repeats, and/or other types of substitutions.

In certain embodiments, the polypeptides described herein are isolated. In certain embodiments, the polypeptides described herein are substantially pure.

The polypeptides, analogs and variants thereof, can be further modified to contain additional chemical moieties not normally part of the polypeptide. The derivatized moieties can improve or otherwise modulate the solubility, the biological half-life, and/or absorption of the polypeptide. The moieties can also reduce or eliminate undesirable side effects of the polypeptides and variants. An overview for chemical moieties can be found in Remington: The Science and Practice of Pharmacy, 22^{st} Edition, 2012, Pharmaceutical Press, London.

The polypeptides described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof.

In some embodiments, a DNA sequence encoding a polypeptide of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding antibodies, or fragments thereof, against human NOTCH3. For example, recombinant expression vectors can be replicable DNA constructs which have synthetic or cDNA-derived DNA fragments encoding a polypeptide chain of an anti-NOTCH3 antibody, or fragment thereof, operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, in situations where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

The anti-NOTCH3 antibodies or polypeptides of the present invention can be expressed from one or more vectors. For example, in some embodiments, one heavy chain polypeptide is expressed by one vector and a light chain polypeptide is expressed by a second vector. In some embodiments, a heavy chain polypeptide and a light chain polypeptide is expressed by one vector.

Suitable host cells for expression of an anti-NOTCH3 antibody or polypeptide (or a NOTCH3 protein to use as an antigen) include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts are described in Pouwels et al., 1985, Cloning Vectors: A Laboratory Manual, Elsevier, New York, N.Y. Additional information regarding methods of protein production, including antibody production, can be found, e.g., in U.S. Patent Publication No. 2008/0187954, U.S. Pat. Nos. 6,413,746, 6,660,501; and International Patent Publication No. WO 04/009823.

Various mammalian culture systems may be used to express recombinant polypeptides. Expression of recombinant proteins in mammalian cells may be desirable because these proteins are generally correctly folded, appropriately modified, and biologically functional. Examples of suitable mammalian host cell lines include, but are not limited to, COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art (see, e.g., Luckow and Summers, 1988, Bio/Technology, 6:47).

Thus, the present invention provides cells comprising the anti-NOTCH3 antibodies or polypeptides described herein. In some embodiments, the cells produce the anti-NOTCH3 antibodies described herein. In certain embodiments, the cells produce antibody 122M23. In certain embodiments, the cells produce variants of antibody 122M23. In some embodiments, the cell is a hybridoma cell. In some embodiments, the cell is a mammalian cell. In some embodiments, the cell is a prokaryotic cell. In some embodiments, the cell is an eukaryotic cell.

The proteins produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Affinity chromatography used for purifying immunoglobulins can include Protein A, Protein G, and Protein L chromatography. Isolated proteins can be physically characterized using such techniques as proteolysis, size exclusion chromatography (SEC), mass spectrometry (MS), nuclear magnetic resonance (NMR), isoelectric focusing (IEF), high performance liquid chromatography (HPLC), and x-ray crystallography. The purity of isolated proteins can be determined using techniques known to those of skill in the art, including but not limited to, SDS-PAGE, SEC, capillary gel electrophoresis, IEF, and capillary isoelectric focusing (cIEF).

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant diethylaminoethyl (DEAE) groups. The matrices can be acrylamide, agarose, dextran, cellulose, or other types commonly employed in protein purification. In some embodiments, a cation exchange step can be employed. Suitable cation exchangers include various insoluble matrices comprising sulfopropyl or carboxymethyl groups. In some embodiments, a hydroxyapatite media can be employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, can be employed to further purify a recombinant protein (e.g., a NOTCH3-binding agent). Some or all of the foregoing purification steps, in various combinations, can be employed to provide a homogeneous recombinant protein.

In some embodiments, recombinant protein produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Publication Nos. 2008/0312425, 2008/0177048, and 2009/0187005.

In certain embodiments, the anti-NOTCH3 antibodies or polypeptides can be used in any one of a number of conjugated (i.e. an immunoconjugate or radioconjugate) or non-conjugated forms. In certain embodiments, the antibodies can be used in a non-conjugated form to harness the subject's natural defense mechanisms including complement-dependent cytotoxicity and antibody dependent cellular toxicity to eliminate malignant or cancer cells.

In some embodiments, the anti-NOTCH3 antibody or polypeptide is conjugated to a cytotoxic agent. In some embodiments, the cytotoxic agent is a chemotherapeutic agent including, but not limited to, methotrexate, adriamycin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, non-binding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated antibody. A variety of radionuclides are available for the production of radioconjugated antibodies including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re and ²¹²Bi. Conjugates of an antibody and one or more small molecule toxins, such as calicheamicins, maytansinoids, trichothenes, and CC1065, and the derivatives of these toxins that have toxin activity, can also be used. Conjugates of an antibody and cytotoxic agent may be made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCl), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

### III. Polynucleotides

In certain embodiments, the invention encompasses polynucleotides comprising polynucleotides that encode an antibody or polypeptide (or a fragment of a polypeptide) that specifically binds NOTCH3. The term “polynucleotides that encode a polypeptide” encompasses a polynucleotide which includes only coding sequences for the polypeptide as well as a polynucleotide which includes additional coding and/or non-coding sequences. For example, in some embodiments, the invention provides a polynucleotide comprising a polynucleotide sequence that encodes an antibody to human NOTCH3 or encodes a fragment of such an antibody (e.g., a fragment comprising the antigen-binding site). The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, the polynucleotide comprises a polynucleotide encoding a polypeptide comprising an amino acid sequence selected from the group consisting of: SEQ ID NO:33, SEQ ID NO:34, SEQ ID NO:37, and SEQ ID NO:38. In some embodiments, the polynucleotide comprises a polynucleotide sequence selected from the group consisting of: SEQ ID NO:31, SEQ ID NO:32, SEQ ID NO:35, and SEQ ID NO:36.

In certain embodiments, the polynucleotide comprises a polynucleotide having a nucleotide sequence at least about 80% identical, at least about 85% identical, at least about 90% identical, at least about 95% identical, and in some embodiments, at least about 96%, 97%, 98% or 99% identical to a polynucleotide comprising a sequence selected from the group consisting of: SEQ ID NO:31, SEQ ID NO:32, SEQ ID NO:35, and SEQ ID NO:36. Also provided is a polynucleotide that comprises a polynucleotide that hybridizes to the complement of SEQ ID NO:31, SEQ ID NO:32, SEQ ID NO:35, or SEQ ID NO:36.

In some embodiments, the heavy chain variable region and the light chain variable region of the antibody is encoded by a polynucleotide comprising SEQ ID NO:31 and SEQ ID NO:35. In some embodiments, the heavy chain variable region and the light chain variable region of the antibody is encoded by a polynucleotide comprising SEQ ID NO:32 and SEQ ID NO:36.

In certain embodiments, the polynucleotides comprise the coding sequence for the mature polypeptide fused in the same reading frame to a polynucleotide which aids, for example, in expression and secretion of a polypeptide from a host cell (e.g., a leader sequence which functions as a secretory sequence for controlling transport of a polypeptide from the cell). The polypeptide having a leader sequence is a pre-protein and can have the leader sequence cleaved by the host cell to form the mature form of the polypeptide. The polynucleotides can also encode for a pro-protein which is the mature protein plus additional 5′ amino acid residues. A mature protein having a pro-sequence is a pro-protein and is an inactive form of the protein. Once the pro-sequence is cleaved an active mature protein remains.

In certain embodiments, the polynucleotides comprise the coding sequence for the mature polypeptide fused in the same reading frame to a marker sequence that allows, for example, for purification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. In some embodiments, the marker sequence is a FLAG-tag, a peptide of sequence DYKDDDDK (SEQ ID NO:46) which can be used in conjunction with other affinity tags.

The present invention further relates to variants of the hereinabove described polynucleotides encoding, for example, fragments, analogs, and/or derivatives.

In certain embodiments, the present invention provides polynucleotides comprising polynucleotides having a nucleotide sequence at least about 80% identical, at least about 85% identical, at least about 90% identical, at least about 95% identical, and in some embodiments, at least about 96%, 97%, 98% or 99% identical to a polynucleotide encoding a polypeptide comprising a anti-NOTCH3 antibody, or fragment thereof, described herein.

As used herein, the phrase a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, a polynucleotide variant contains alterations which produce silent substitutions, additions, or deletions, but does not alter the properties or activities of the encoded polypeptide. In some embodiments, a polynucleotide variant comprises silent substitutions that result in no change to the amino acid sequence of the polypeptide (due to the degeneracy of the genetic code). In some embodiments, nucleotide variants comprise nucleotide sequences which result in expression differences (e.g., increased or decreased expression) at the transcript level. Polynucleotide variants can be produced for a variety of reasons, for example, to optimize codon expression for a particular host (i.e., change codons in the human mRNA to those preferred by a bacterial host such as E. coli). In some embodiments, a polynucleotide variant comprises at least one silent mutation in a non-coding or a coding region of the sequence.

In some embodiments, a polynucleotide variant is produced to modulate or alter expression (or expression levels) of the encoded polypeptide. In some embodiments, a polynucleotide variant is produced to increase expression of the encoded polypeptide. In some embodiments, a polynucleotide variant is produced to decrease expression of the encoded polypeptide. In some embodiments, a polynucleotide variant has increased expression of the encoded polypeptide as compared to a parental polynucleotide sequence. In some embodiments, a polynucleotide variant has decreased expression of the encoded polypeptide as compared to a parental polynucleotide sequence.

In certain embodiments, the polynucleotides are isolated. In certain embodiments, the polynucleotides are substantially pure.

Vectors comprising the polynucleotides described herein are also provided. Cells comprising the polynucleotides described herein are also provided. In some embodiments, an expression vector comprises a polynucleotide molecule. In some embodiments, a host cell comprises an expression vector comprising the polynucleotide molecule. In some embodiments, a host cell comprises a polynucleotide molecule.

### IV. Methods of Use

As shown herein, the sensitivity of human pancreatic tumors to the anti-NOTCH2/3 antibody OMP-59R5 significantly correlated with increased NOTCH3 expression at both the gene and protein levels. The correlation between high levels of NOTCH3 expression and the responsiveness of pancreatic tumors to the anti-NOTCH2/3 antibody OMP-59R5 can be exploited to improve methods of treating pancreatic cancer. Selecting pancreatic cancer patients for treatment with the anti-NOTCH2/3 antibody OMP-59R5 whose tumors are determined to likely be responsive to treatment based on the NOTCH3 expression level should increase overall therapeutic value. Therapeutic efficacy can also be improved by not selecting pancreatic cancer patients for OMP-59R5 therapy whose tumors are determined to likely be non-responsive to treatment.

Provided herein are methods for detecting NOTCH3 expression in tumor samples. Provided herein are methods for determining the level of NOCTH3 expression in tumor samples.

In some embodiments, the samples include, but are not limited to, any clinically relevant tissue sample, such as a tumor biopsy, a core biopsy tissue sample, a fine needle aspirate, a hair follicle, or a sample of bodily fluid, such as blood, plasma, serum, lymph, ascites fluid, cystic fluid, or urine. In some embodiments, the sample is taken from a patient having a pancreatic tumor or cancer. In some embodiments, the sample is a primary tumor. In some embodiments, the sample is a metastasis. The sample may be taken from a human, or from non-human mammals such as, mice, rats, non-human primates, canines, felines, ruminants, swine, or sheep. In some embodiments, samples are taken from a subject at multiple time points, for example, before treatment, during treatment, and/or after treatment. In some embodiments, samples are taken from different locations in the subject, for example, a sample from a primary tumor and a sample from a metastasis in a distant location.

In some embodiments, the tumor sample is a lung tumor, a small-cell lung tumor, a non-small cell lung tumor, a hepatocellular tumor, a gastrointestinal tumor, a pancreatic tumor, a glioblastoma, a cervical cancer tumor, an ovarian tumor, a liver tumor, a bladder tumor, hepatoma, a breast tumor, a colon tumor, a colorectal tumor, an endometrial or uterine tumor, a salivary gland tumor, a kidney tumor, a prostate tumor, a thyroid tumor, or a head and neck tumor.

In some embodiments, the sample is a paraffin-embedded fixed tissue sample. In some embodiments, the sample is a formalin-fixed paraffin embedded (FFPE) tissue sample. In some embodiments, the sample is a fresh tissue (e.g., tumor) sample. In some embodiments, the sample is a frozen tissue sample. In some embodiments, the sample is a fresh frozen (FF) tissue (e.g., tumor) sample. In some embodiments, the sample is a cell isolated from a fluid. In some embodiments, the sample comprises circulating tumor cells (CTCs). In some embodiments, the sample is an archival tissue sample. In some embodiments, the sample is an archival tissue sample with known diagnosis, treatment, and/or outcome history. In some embodiments, the sample is a block of tissue. In some embodiments, the sample is dispersed cells. In some embodiments, the sample size is from about 1 cell to about 1×10⁶ cells or more. In some embodiments, the sample size is about 10 cells to about 1×10⁵ cells. In some embodiments, the sample size is about 10 cells to about 10,000 cells. In some embodiments, the sample size is about 10 cells to about 1,000 cells. In some embodiments, the sample size is about 10 cells to about 100 cells. In some embodiments, the sample size is about 1 cell to about 10 cells. In some embodiments, the sample size is a single cell.

In some embodiments, NOTCH3 expression is analyzed by assessing protein expression as compared to gene expression. Commonly used methods for the analysis of protein expression, include but are not limited to, immunohistochemistry (IHC)-based, antibody-based, and mass spectrometry-based methods. Antibodies, generally monoclonal antibodies, may be used to detect expression of a gene product (e.g., protein). In some embodiments, the antibodies can be detected by direct labeling of the antibodies themselves. In other embodiments, an unlabeled primary antibody is used in conjunction with a labeled secondary antibody.

In some embodiments, NOTCH3 expression is determined by an assay known to those of skill in the art, including but not limited to, multi-analyte profile test, enzyme-linked immunosorbent assay (ELISA), radioimmunoassay, Western blot assay, immunofluorescent assay, enzyme immunoassay, immunoprecipitation assay, chemiluminescent assay, immunohistochemical (IHC) assay, dot blot assay or slot blot assay. In some embodiments, NOTCH3 expression is determined by an IHC assay.

In some embodiments, NOTCH3 expression is determined using an agent that specifically binds to NOTCH3. Any molecular entity that displays specific binding to NOTCH3 can be employed to determine the level of NOTCH3 protein in a sample. Specific binding agents include, but are not limited to, antibodies, antibody mimetics, and polynucleotides (e.g., aptamers). One of skill understands that the degree of specificity required is determined by the particular assay used to detect NOTCH3 protein. In some embodiments, the agent used to detect and/or determine NOTCH3 expression is an anti-NOTCH3 antibody described herein.

In some embodiments, wherein an antibody is used in the assay the antibody is detectably labeled. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin; and examples of suitable radioactive material include ¹²⁵I, ¹³¹I, ³⁵S, or ³H.

In some embodiments, NOTCH3 expression is determined using an IHC assay. For example, 4 μm-thick FFPE sections are cut from a tumor sample and mounted on coated glass slides. Tissues are deparaffinized and rehydrated by successively incubating them in xylene, 100% ethanol, 95% ethanol, 70% ethanol, and distilled water for antigen retrieval. Slides are placed into retrieval solution and placed in a decloaker for antigen retrieval. To block endogenous peroxidase activity slides are incubated in 6% hydrogen peroxide for 5 minutes and washed in PBS. To block non-specific background staining slides are incubated in blocker for 30 minutes at room temperature. Slides are incubated with an anti-NOTCH3 antibody overnight at 4° C. Specific binding is detected using a kit including diaminobenzidine (DAB). The sections are counterstained with hematoxylin. The slides may be analyzed using an automated instrument or evaluated manually by microscope. The staining intensity of each tumor cell (0: no expression, 1: weak expression, 2: moderate expression, 3: strong expression) is measured and cells of each staining level are counted and a percentage for each type is calculated. The data is combined into a weighted H-score for each tissue section: H-score=[3×(% 3+cells)]+[2×(% 2+cells)]+[1×(% 1+cells)]. Using these parameters, the highest score available is H-score=300. In some embodiments, the antibody used in an IHC assay is an anti-NOTCH3 antibody described herein. In some embodiments, the antibody is 122M23.

Other suitable methods for analyzing NOTCH3 expression include proteomics-based methods. Proteomics includes, among other things, study of the global changes of protein expression in a sample. In some embodiments, a proteomic method comprises the following steps: (1) separation of individual proteins in a sample by 2-D electrophoresis (2-D PAGE), (2) identification of individual proteins recovered from the gel (e.g., by mass spectrometry or N-terminal sequencing), and (3) analysis of the data using bioinformatics. In some embodiments, a proteomic method comprises using a tissue microarray (TMA). Tissue arrays may be constructed according to a variety of techniques known to one of skill in the art. In certain embodiments, a manual tissue arrayer is used to remove a “core” from a paraffin block prepared from a tissue sample. The core is then inserted into a separate paraffin block in a designated location on a grid. Cores from as many as about 400 samples can be inserted into a single recipient block. The resulting tissue array may be processed into thin sections for analysis. In some embodiments, a proteomic method comprises an antibody microarray. In some embodiments, a proteomic method comprises using mass spectrometry, including but not limited to, SELDI, MALDI, electro spray, and surface plasmon resonance methods. In some embodiments, a proteomic method comprises bead-based technology, including but not limited to, antibodies on beads in an array format. In some embodiments, the proteomic method comprises a reverse phase protein microarray (RPPM). In some embodiments, the proteomic method comprises multiplexed protein profiling, including but not limited to, the Global Proteome Survey (GPS) method.

In some embodiments of any of the methods or uses described herein, classification probabilities of a tumor (in regard to responder or non-responder status) are obtained based on the decision values. In some embodiments, the probabilities are obtained by fitting a logistic regression on the decision values.

Provided herein are methods for identifying, selecting, and/or stratifying tumors and/or patients with pancreatic cancer that are likely to be responsive (“sensitive”) or non-responsive (“resistant”) to treatment with a NOTCH pathway inhibitor, particularly the anti-NOTCH2/3 antibody 59R5. In addition, provided are methods for treating patients with cancer who are likely to respond to treatment, are predicted to respond to treatment, and/or have been identified to respond to treatment with a NOTCH inhibitor.

In some embodiments, methods of identifying a pancreatic tumor likely to be responsive to a first antibody that specifically binds human NOTCH2/3 are provided. In some embodiments, a method comprises determining the level of NOTCH3 expression in a sample obtained from the pancreatic tumor, wherein the level of NOTCH3 expression is determined using a second antibody that specifically binds the extracellular domain of human NOTCH3. In some embodiments, the second antibody is an anti-NOTCH3 antibody described herein. In some embodiments, the second antibody comprises a heavy chain CDR1 comprising TKYTIH (SEQ ID NO:39), a heavy chain CDR2 comprising YINPSNDYTNYNQTFKD (SEQ ID NO:40), a heavy chain CDR3 comprising ARGTTPYSLDY (SEQ ID NO:41); a light chain CDR1 comprising RASESVDNYGMSFMN (SEQ ID NO:42), a light chain CDR2 comprising AASNQGS (SEQ ID NO:43), and a light chain CDR3 comprising LQSKEVP (SEQ ID NO:44).

In some embodiments, methods of identifying a subject with a pancreatic tumor as likely to be responsive to a first antibody that specifically binds human NOTCH2/3 are provided. In some embodiments, a method comprises (a) determining the level of NOTCH3 expression in a sample obtained from the pancreatic tumor, wherein the level of NOTCH3 expression is determined using a second antibody that specifically binds the extracellular domain of human NOTCH3. In some embodiments, the second antibody is an anti-NOTCH3 antibody described herein. In some embodiments, the second antibody comprises a heavy chain CDR1 comprising TKYTIH (SEQ ID NO:39), a heavy chain CDR2 comprising YINPSNDYTNYNQTFKD (SEQ ID NO:40), a heavy chain CDR3 comprising ARGTTPYSLDY (SEQ ID NO:41); a light chain CDR1 comprising RASESVDNYGMSFMN (SEQ ID NO:42), a light chain CDR2 comprising AASNQGS (SEQ ID NO:43), and a light chain CDR3 comprising LQSKEVP (SEQ ID NO:44); and (b) identifying the subject as likely to be responsive or non-responsive to treatment with the first antibody that specifically binds human NOTCH2/3 based upon the level of NOTCH3 expression in the pancreatic tumor.

In some embodiments, methods for selecting a subject with a pancreatic tumor for treatment with a first antibody that specifically binds human NOTCH2/3 are provided. In some embodiments, a method comprises (a) determining the level of NOTCH3 expression in a sample obtained from the pancreatic tumor, wherein the level of NOTCH3 expression is determined using a second antibody that specifically binds the extracellular domain of human NOTCH3. In some embodiments, the second antibody is an anti-NOTCH3 antibody described herein. In some embodiments, the second antibody comprises a heavy chain CDR1 comprising TKYTIH (SEQ ID NO:39), a heavy chain CDR2 comprising YINPSNDYTNYNQTFKD (SEQ ID NO:40), a heavy chain CDR3 comprising ARGTTPYSLDY (SEQ ID NO:41); a light chain CDR1 comprising RASESVDNYGMSFMN (SEQ ID NO:42), a light chain CDR2 comprising AASNQGS (SEQ ID NO:43), and a light chain CDR3 comprising LQSKEVP (SEQ ID NO:44); (b) identifying the pancreatic tumor as likely to be responsive or non-responsive to treatment with the first antibody that specifically binds human NOTCH2/3 based upon the level of NOTCH3 expression in the pancreatic tumor; and (c) selecting the subject for treatment with the first antibody if the pancreatic tumor is identified as likely to be responsive to treatment.

In some embodiments, methods for stratifying a pancreatic cancer patient population for treatment with a first antibody that specifically binds human NOTCH2/3 are provided. In some embodiments, a method comprises: (a) determining the level of NOTCH3 expression samples from the patients, wherein the level of NOTCH3 expression is determined using a second antibody that specifically binds the extracellular domain of human NOTCH3. In some embodiments, the second antibody is an anti-NOTCH3 antibody described herein. In some embodiments, the second antibody comprises a heavy chain CDR1 comprising TKYTIH (SEQ ID NO:39), a heavy chain CDR2 comprising YINPSNDYTNYNQTFKD (SEQ ID NO:40), a heavy chain CDR3 comprising ARGTTPYSLDY (SEQ ID NO:41); a light chain CDR1 comprising RASESVDNYGMSFMN (SEQ ID NO:42), a light chain CDR2 comprising AASNQGS (SEQ ID NO:43), and a light chain CDR3 comprising LQSKEVP (SEQ ID NO:44); (b) stratifying the patient population based on the expression level of NOTCH3 in the samples.

In some embodiments, the pancreatic cancer is an exocrine tumor of the pancreas. In some embodiments, the pancreatic cancer is acinar cell carcinoma, adenocarcinoma, adenosquamous carcinoma, giant cell tumor, intraductal papillary-mucinous neoplasm (IPMN), mucinous cystadenocarcinoma, pancreatoblastoma, serous cystadenocarcinoma, or solid and pseudopapillary tumor. In some embodiments, the pancreatic cancer treated is adenocarcinoma. In some embodiments, the pancreatic cancer is a neuroendocrine tumor. In some embodiments, the pancreatic neuroendocrine tumor is a gastrinoma, glucagonoma, insulinoma, nonfunctional islet cell tumor, VIPoma, or somatostatinoma. In some embodiments, the pancreatic cancer is not a neuroendocrine tumor.

In some embodiments, the pancreatic cancer is a resectable tumor, a locally advanced cancer, or a metastatic pancreatic cancer. In some embodiments, the pancreatic cancer is a grade 1, 2, 3 or 4 cancer as determined according to the stages of the AJCC TNM system.

In some embodiments of the methods described herein, a patient is selected for treatment with an antibody that specifically binds human NOTCH2/3. In certain embodiments, the NOTCH2/3 antibody comprises a heavy chain CDR1 comprising SSSGMS (SEQ ID NO:1), a heavy chain CDR2 comprising VIASSGSNTYYADSVKG (SEQ ID NO:2), and a heavy chain CDR3 comprising SIFYTT (SEQ ID NO:4), and a light chain CDR1 comprising RASQSVRNYLA (SEQ ID NO:12), a light chain CDR2 comprising GASSRAT (SEQ ID NO:13), and a light chain CDR3 comprising QQYSNFPI (SEQ ID NO:14). In certain embodiments, the NOTCH2/3 antibody comprises a heavy chain variable region comprising the amino acids of SEQ ID NO:18. In certain embodiments, the NOTCH2/3 antibody further comprises a light chain variable region comprising the amino acids of SEQ ID NO:26. In certain embodiments, the NOTCH2/3 antibody comprises the same heavy and light chain amino acid sequences as an antibody encoded by a plasmid deposited with ATCC having deposit no. PTA-10170. In certain embodiments, the NOTCH2/3 antibody is encoded by the plasmid having ATCC deposit no. PTA-10170 which was deposited with the American Type Culture Collection (ATCC), at 10801 University Boulevard, Manassas, Va., 20110, under the conditions of the Budapest Treaty on Jul. 6, 2009. In certain embodiments, the NOTCH2/3 antibody competes for specific binding to human NOTCH2 or human NOTCH3 with an antibody encoded by the plasmid deposited with ATCC having deposit no. PTA-10170 or PTA-9547. PTA-9547 was deposited with the ATCC under the conditions of the Budapest Treaty on Oct. 15, 2008. In certain embodiments, the NOTCH2/3 antibody is OMP-59R5.

In certain embodiments, in addition to administering an antibody that specifically binds human NOTCH2/3 (e.g. OMP-59R5), the method or treatment further comprises administering at least one additional therapeutic agent. An additional therapeutic agent can be administered prior to, concurrently with, and/or subsequently to, administration of the antibody that specifically binds human NOTCH2/3. Pharmaceutical compositions comprising an antibody that specifically binds human NOTCH2/3 and the additional therapeutic agent(s) are also provided. In some embodiments, the at least one additional therapeutic agent comprises 1, 2, 3, or more additional therapeutic agents.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). Combination therapy may decrease the likelihood that resistant cancer cells will develop. In some embodiments, combination therapy comprises a therapeutic agent that affects (e.g., inhibits or kills) non-tumorigenic cells and a therapeutic agent that affects (e.g., inhibits or kills) tumorigenic CSCs.

In some embodiments, the combination of an antibody that specifically binds human NOTCH2/3 and at least one additional therapeutic agent results in additive or synergistic results. In some embodiments, the combination therapy results in an increase in the therapeutic index of the antibody that specifically binds human NOTCH2/3. In some embodiments, the combination therapy results in an increase in the therapeutic index of the additional agent(s). In some embodiments, the combination therapy results in a decrease in the toxicity and/or side effects of the antibody that specifically binds human NOTCH2/3. In some embodiments, the combination therapy results in a decrease in the toxicity and/or side effects of the additional agent(s).

Useful classes of therapeutic agents include, for example, anti-tubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, anti-folates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an alkylating agent, an antimetabolite, an antimitotic, a topoisomerase inhibitor, or an angiogenesis inhibitor. In some embodiments, the second therapeutic agent is a platinum complex such as carboplatin or cisplatin. In some embodiments, the additional therapeutic agent is a platinum complex in combination with a taxane.

Therapeutic agents that may be administered in combination with an antibody that specifically binds human NOTCH2/3 include chemotherapeutic agents. Thus, in some embodiments, the method or treatment involves the administration of an antibody that specifically binds human NOTCH2/3 in combination with a chemotherapeutic agent or cocktail of multiple different chemotherapeutic agents. Treatment with an antibody that specifically binds human NOTCH2/3 can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in The Chemotherapy Source Book, 4^{th} Edition, 2008, M. C. Perry, Editor, Lippincott, Williams & Wilkins, Philadelphia, Pa.

Chemotherapeutic agents useful in the instant invention include, but are not limited to, alkylating agents such as thiotepa and cyclophosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel and docetaxel; chlorambucil; gemcitabine; 6-thioguanine; mercaptopurine; platinum analogs such as cisplatin and carboplatin; vinblastine; platinum; etoposide; ifosfamide; mitomycin C; mitoxantrone; vincristine; vinorelbine; navelbine; novantrone; teniposide; daunomycin; aminopterin; xeloda; ibandronate; CPT11; topoisomerase inhibitor RFS 2000; difluoromethylornithine; retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In some embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide, and irinotecan, as well as pharmaceutically acceptable salts, acids, or derivatives of any of these.

In some embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, raltitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these. In some embodiments, a method described herein comprises administering to a pancreatic cancer patient a therapeutically effective amount of the OMP-59R5 antibody in combination with an anti-metabolite. In some embodiments, the anti-metabolite is a nucleoside analogue. In some embodiments, a method described herein comprises administering to a pancreatic cancer patient a therapeutically effective amount of the OMP-59R5 antibody in combination with gemcitabine.

In some embodiments, the chemotherapeutic agent is an antimitotic agent, including, but not limited to, agents that bind tubulin. In some embodiments, the agent is a taxane. In some embodiments, the agent is paclitaxel or docetaxel, or a pharmaceutically acceptable salt, acid, or derivative of paclitaxel or docetaxel. In some alternative embodiments, the antimitotic agent comprises a vinca alkaloid, such as vincristine, vinblastine, vinorelbine, or vindesine, or pharmaceutically acceptable salts, acids, or derivatives thereof. In some embodiments, a method described herein comprises administering to a pancreatic cancer patient a therapeutically effective amount of the OMP-59R5 antibody in combination with an antimitotic agent. In some embodiments, the anti-metabolite is a taxane. In some embodiments, a method described herein comprises administering to a pancreatic cancer patient a therapeutically effective amount of the OMP-59R5 antibody in combination with ABRAXANE™ (albumin-bound paclitaxel).

In some embodiments, the treatment involves the combined administration of an antibody that specifically binds human NOTCH2/3 (e.g., OMP-59R5) and radiation therapy. Treatment with the antibody that specifically binds human NOTCH2/3 (e.g., OMP-59R5) can occur prior to, concurrently with, or subsequent to administration of radiation therapy. Dosing schedules for such radiation therapy can be determined by the skilled medical practitioner. In some embodiments, the antibody that specifically binds human NOTCH2/3 (e.g., OMP-59R5) is administered after radiation treatment. In some embodiments, the antibody that specifically binds human NOTCH2/3 (e.g., OMP-59R5) is administered with radiation therapy.

In some embodiments, a second therapeutic agent comprises an antibody. Thus, treatment can involve the combined administration of an antibody that specifically binds human NOTCH2/3 (e.g., OMP-59R5) with other antibodies against additional tumor-associated antigens including, but not limited to, antibodies that bind to EGFR, ErbB2, DLL4, or NF-κB. Exemplary anti-DLL4 antibodies are described, for example, in U.S. Pat. No. 7,750,124. Additional anti-DLL4 antibodies are described in, e.g., International Patent Pub. Nos. WO 2008/091222 and WO 2008/0793326, and U.S. Patent Application Pub. Nos. 2008/0014196; 2008/0175847; 2008/0181899; and 2008/0107648.

Combined administration can include co-administration, either in a single pharmaceutical formulation or using separate formulations, or consecutive administration in either order but generally within a time period such that all active agents can exert their biological activities simultaneously.

Treatment with an antibody that specifically binds human NOTCH2/3 (e.g., OMP-59R5) can include combination treatment with one or more cytokines (e.g., lymphokines, interleukins, tumor necrosis factors, and/or growth factors) or can be accompanied by surgical removal of tumors, cancer cells or any other therapy deemed necessary by a treating physician.

### V. Kits

The present invention provides kits that comprise the anti-NOTCH3 antibodies described herein. In certain embodiments, a kit comprises at least one purified antibody against human NOTCH3 in one or more containers. In some embodiments, the kits contain all of the components necessary and/or sufficient to perform a detection assay (i.e. an IHC assay), including all controls, directions for performing assays, and any necessary software for analysis and presentation of results.

It will be further appreciated that any or all steps in the methods of the invention could be implemented by personnel or, alternatively, performed in an automated fashion. Thus, the steps of body sample preparation, sample freezing or fixing, RNA extraction, and/or detection of NOTCH3 transcript level can be automated.

Embodiments of the present disclosure can be further defined by reference to the following non-limiting examples, which describe in detail preparation of certain antibodies of the present disclosure and methods for using antibodies of the present disclosure. It will be apparent to those skilled in the art that many modifications, both to materials and methods, may be practiced without departing from the scope of the present disclosure.

### EXAMPLES

### Example 1

### Inhibition of Pancreatic Tumor Growth In Vivo by Anti-NOTCH2/3 Antibody 59R5 in Combination with Gemcitabine

Ten primary patient-derived pancreatic tumor xenografts were tested for efficacy in response to anti-Notch2/3 antibody OMP-59R5 in combination with gemcitabine. These tumors were OMP-PN04, OMP-PN07, OMP-PN08, OMP-PN11, OMP-PN13, OMP-PN16, OMP-PN17, OMP-PN21, OMP-PN23, and OMP-PN25.

In one representative example, OMP-PN8 pancreatic tumor cells (2×10⁴) were injected into NOD/SCID mice. Tumors were allowed to grow for 22 days until they reached an average volume of 125 mm³. Tumor-bearing mice were randomized into 4 groups and treated with control antibody, anti-NOTCH2/3 antibody OMP-59R5, gemcitabine, or a combination of OMP-59R5 and gemcitabine. Antibodies were dosed every other week at 40 mg/kg. Gemcitabine was dosed weekly at 20 mg/kg. Tumor growth was monitored and tumor volumes were measured with electronic calipers on the indicated days post-treatment.

In a second representative example, OMP-PN11 pancreatic tumor cells (5×10⁴) were injected into NOD/SCID mice. Tumors were allowed to grow for 21 days until they reached an average volume of 120 mm³. Tumor-bearing mice were randomized into 4 groups and treated with control antibody, OMP-59R5, gemcitabine, or a combination of OMP-59R5 and gemcitabine. Antibodies were dosed every other week at 40 mg/kg. Gemcitabine was dosed weekly at 20 mg/kg. Tumor volumes were measured on the indicated days post-treatment. Tumor growth was monitored and tumor volumes were measured with electronic calipers on the indicated days post-treatment.

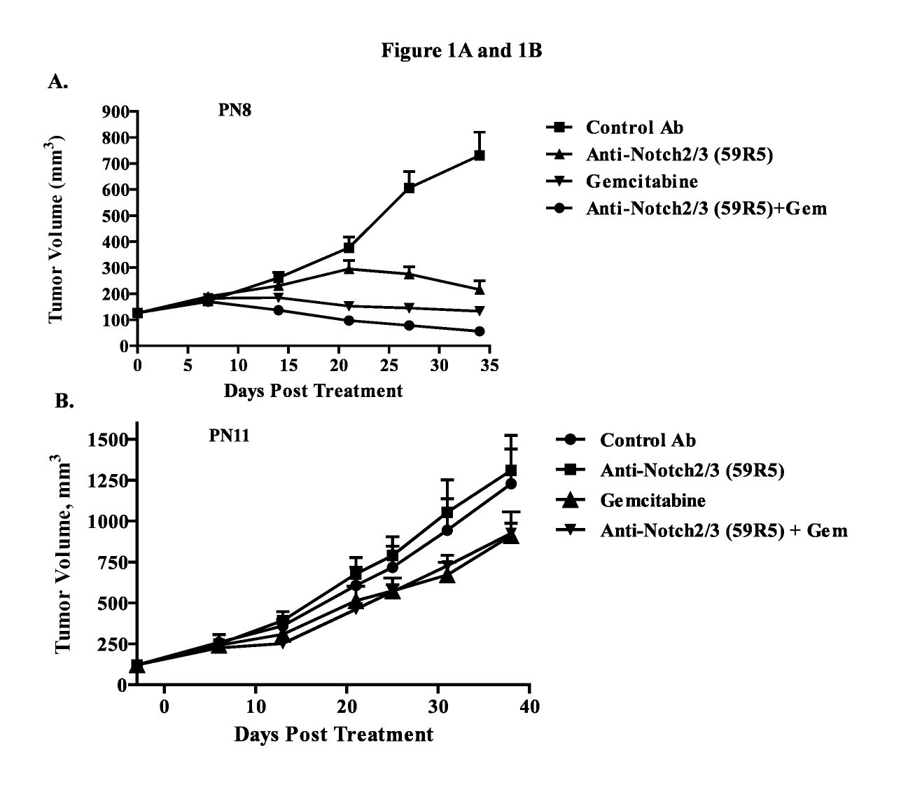

As shown in FIG. 1A, OMP-59R5 significantly inhibited tumor growth both as a single agent and in combination with gemcitabine. In contrast, OMP-59R5 had no effect on OMP-PN11 tumor growth either as a single agent or in combination with gemcitabine (FIG. 1B). A summary of the results for the 10 pancreatic tumors is found in Table 2. The results shown are for efficacy with 59R5 in combination with gemcitabine.

TABLE 2

| Pancreatic Tumor | Efficacy |
|---|---|
| OMP-PN04 | + |
| OMP-PN07 | − |
| OMP-PN08 | + |
| OMP-PN11 | − |
| OMP-PN13 | − |
| OMP-PN16 | + |
| OMP-PN17 | + |
| OMP-PN21 | + |
| OMP-PN23 | − |
| OMP-PN25 | + |

### Example 2

### NOTCH2 and NOTCH3 Expression Levels

Using standard microarray technology NOTCH2 and NOTCH3 expression levels were determined in the ten pancreatic tumors described in Example 1. Expression data were obtained using Affymetrix® U133 plus 2 arrays according to the manufacturer's instructions. The results are shown in Table 3. NOTCH2 and NOTCH3 expression levels of each pancreatic tumor were analyzed in correlation to the in vivo responsiveness of the pancreatic tumors to treatment with 59R5 in combination with gemcitabine. These analyses could identify if NOTCH2 and/or NOTCH3 could be used as a predictive biomarker. The analyses of NOTCH2 and NOTCH3 expression levels shown in Table 3 were based on a cut-off value of 500. However, the overall conclusion from the analyses remained the same when the cut-off value was varied between 300 and 1000. Surprisingly, in pancreatic tumors there was a very strong correlation between high levels of NOTCH3 expression and the responsiveness of the tumors to treatment with OMP-59R5 in combination with gemcitabine. Seven of the 10 pancreatic tumors had high NOTCH3 expression and six of these pancreatic tumors were responsive to treatment with OMP-59R5 and gemcitabine. All three of the pancreatic tumors that had low NOTCH3 expression were non-responsive to treatment with 59R5 in combination with gemcitabine. No correlation between NOTCH2 expression and in vivo responsiveness was observed in the ten pancreatic tumors.

TABLE 3

| Tumor | NOTCH3 expression | NOTCH2 expression |
|---|---|---|
| OMP-PN04 | High (1802) | High (4637) |
| OMP-PN07 | Low (274) | High (2140) |
| OMP-PN08 | High (2484) | High (6909) |
| OMP-PN11 | Low (141) | High (4576) |
| OMP-PN13 | Low (23) | High (6848) |
| OMP-PN16 | High (3318) | High (3812) |
| OMP-PN17 | High (6106) | High (5904) |
| OMP-PN21 | High (2776) | High (6203) |
| OMP-PN23 | High (2978) | High (5166) |
| OMP-PN25 | High (6600) | High (4383) |

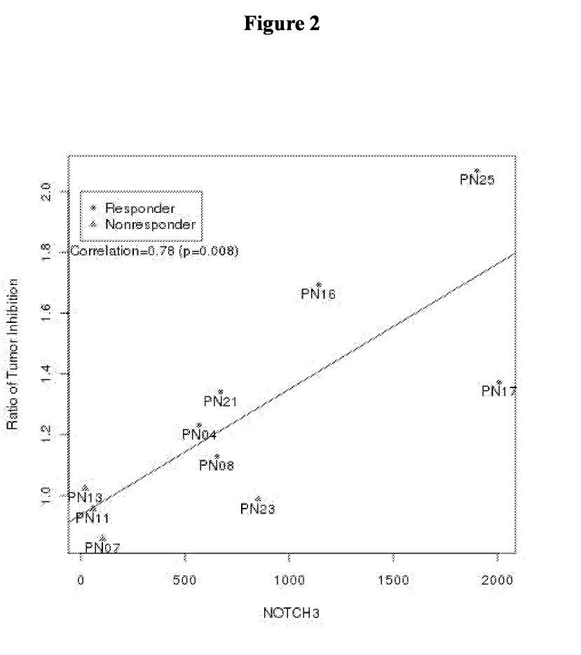

The correlation between high levels of NOTCH3 expression and the responsiveness of pancreatic tumors to treatment with OMP-59R5 in combination with gemcitabine was further analyzed. NOTCH3 gene expression levels were determined in the OMP-PN04, OMP-PN08, OMP-PN11, OMP-PN13, OMP-PN16, OMP-PN17, OMP-PN21, OMP-PN23, and OMP-PN25 pancreatic tumor cells using standard multiplex transcript sequencing (e.g., RNASeq). RNASeq was performed using the Illumina® HiSeq™ 2000 Sequencing System according to the manufacturer's instructions. FIG. 2 shows that increased NOTCH3 expression significantly correlated (0.823; p<0.021) with in vivo tumor inhibition by OMP-59R5 in combination with gemcitabine treatment in human pancreatic xenograft models.

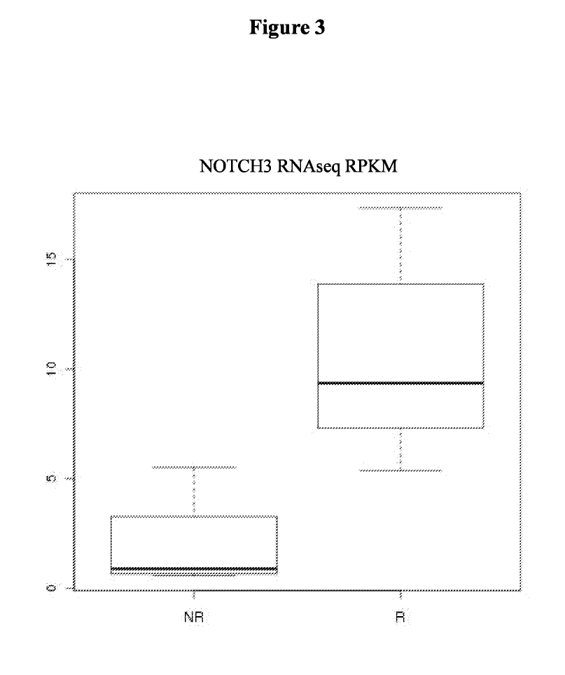

FIG. 3 shows the distribution of NOTCH3 expression detected in xenografts of human pancreatic tumors which were responsive to treatment with OMP-59R5 anti-NOTCH2/3 antibody in combination with gemcitabine (R=responders: pval<0.05 compared to gemcitabine treatment alone) and for those xenografts which were found to be non-responsive to treatment with OMP-59R5 anti-NOTCH2/3 antibody in combination with gemcitabine (NR=non-responders: pval>0.05 compared to gemcitabine treatment alone). The distribution of NOTCH3 expression levels in non-responsive pancreatic tumors showed a clear separation from the distribution of NOTCH3 expression levels in responsive pancreatic tumors.

### Example 3

### Anti-NOTCH3 Monoclonal Antibody as Immunohistochemistry Reagent

Immunohistochemistry (IHC) is the localization of antigens in tissue sections by the use of specific antibodies through antigen-antibody interactions that are then visualized and detected. However, finding an antibody that works well in an IHC assay, particularly on formalin-fixed paraffin embedded (FFPE) material, can be a difficult endeavor. Common problems include, but are not limited to, strong background staining and weak antigen staining. Many antibodies that work very well in fluorescence-activated cell sorting (FACS) assays, ELISAs, or Western blot analyses have undesirable characteristics in an IHC assay. Therefore an extensive screening project was undertaken to identify an antibody that specifically bound the extracellular domain of human NOTCH3 that would be appropriate for use in IHC assays. In addition to evaluating commercially available anti-NOTCH3 antibodies, murine anti-NOTCH3 monoclonal antibodies were produced and evaluated.

Antibodies were generated against recombinant human NOTCH3 protein amino acids 350-580 (NOTCH3 EGFs 9-14). Mice (n=3) were immunized with NOTCH3 protein using standard techniques. Sera from individual mice were screened against NOTCH3 approximately 70 days after initial immunization using FACS analysis. The animal with the highest antibody titer was selected for final antigen boost after which spleen cells were isolated for hybridoma production. SP2/0 cells were used as fusion partners for the mouse spleen cells. Hybridoma cells were plated at 1 cell per well in 96 well plates, and the supernatants were screened against human NOTCH3 ECD.

Screening was first done by FACS, followed by Western blot analyses and then in IHC assays using HEK-293 cells transiently transfected with NOTCH1, NOTCH2, NOTCH3, or NOTCH4 ECD constructs. Starting with 68 supernatants from hybridoma cells, seven candidate antibodies were selected for further testing and optimization in IHC assays, 122M23, 122M25, 122M26, 122M27, 122M35, 122M36, and 122M40. In addition to the seven 122 series antibodies, at least 11 commercially available anti-NOTCH3 antibodies were screened in IHC assays. The desired antibody needed to be specific for NOTCH3 ECD, so the staining had to be predominantly membrane localized, with minimal or no detectable background non-specific staining in transiently transfected cell pellets in FFPE format. In addition, the antibody had to give appropriate results on positive and negative control tissues.

None of the commercially available anti-NOTCH3 antibodies gave satisfactory staining in the IHC assay. One antibody from the anti-NOTCH3 hybridoma series, 122M23, was identified that met all of the desired criteria and was further characterized by sequencing. A plasmid encoding the heavy chain of antibody 122M23 was deposited with the ATCC, 10801 University Boulevard, Manassas, Va., USA, under the conditions of the Budapest Treaty on Apr. 4, 2014 and assigned ATCC deposit designation number PTA-121156. A plasmid encoding the light chain of antibody 122M23 was deposited with the ATCC, 10801 University Boulevard, Manassas, Va., USA, under the conditions of the Budapest Treaty on Apr. 4, 2014 and assigned ATCC deposit designation number PTA-121155. A hybridoma producing the antibody 122M23 was deposited with the ATCC, 10801 University Boulevard, Manassas, Va., USA, under the conditions of the Budapest Treaty on Apr. 4, 2014 and assigned ATCC deposit designation number PTA-121154. The amino acid sequences of the heavy chain variable region and light chain variable region of 122M23 are SEQ ID NO:34 and SEQ ID NO:38. The nucleotide sequences of the heavy chain variable region and light chain variable region of 122M23 are SEQ ID NO:32 and SEQ ID NO:36. The heavy and light chain CDRs of 122M23 are listed in Table 1 herein.

### Example 4

### NOTCH3 Protein Expression Assessed by IHC

A NOTCH3 immunohistochemistry (IHC) assay was developed and optimized using the murine monoclonal antibody 122M23 identified in Example 3. As described herein antibody 122M23 is specific for the extracellular domain of human NOTCH3. 4 μm-thick FFPE sections were cut and mounted on coated glass slides. Tissues were deparaffinized and rehydrated by successively incubating them in xylene, 100% ethanol, 95% ethanol, 70% ethanol, and distilled water for antigen retrieval. Slides were placed into retrieval solution (Diva, BioCare Medical, Concord Calif.) and placed in a Decloaker (BioCare Medical, Concord Calif.) for antigen retrieval. To block endogenous peroxidase activity slides were incubated in 6% hydrogen peroxide for 5 minutes and washed in PBS. To block non-specific background staining slides were incubated in CAS-Block (Life Technologies) for 30 minutes at room temperature. Slides were incubated with anti-NOTCH3 antibody 122M23 overnight at 4° C. at 12.5 μg/ml. Specific binding was detected using an ADVANCE kit (Dako, Carpinteria Calif.) including diaminobenzidine (DAB) for 5 minutes. The sections were counterstained with hematoxylin.

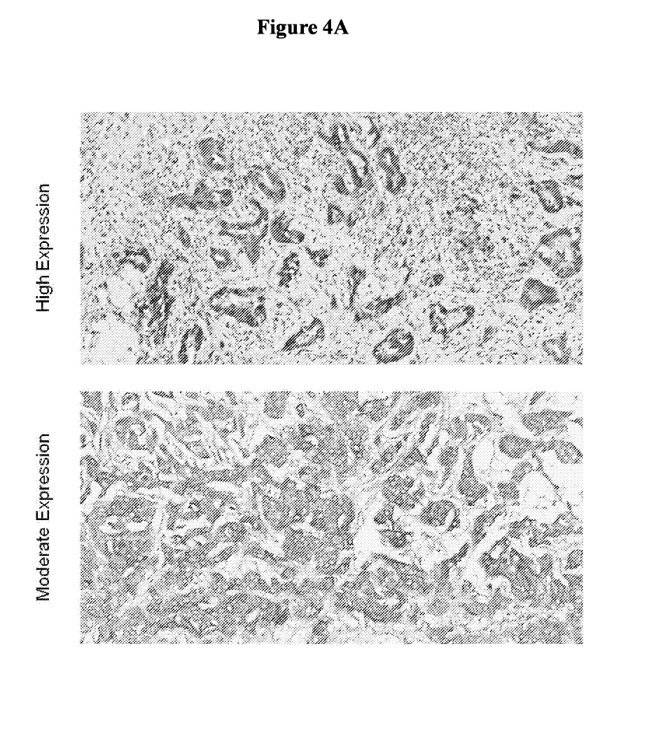

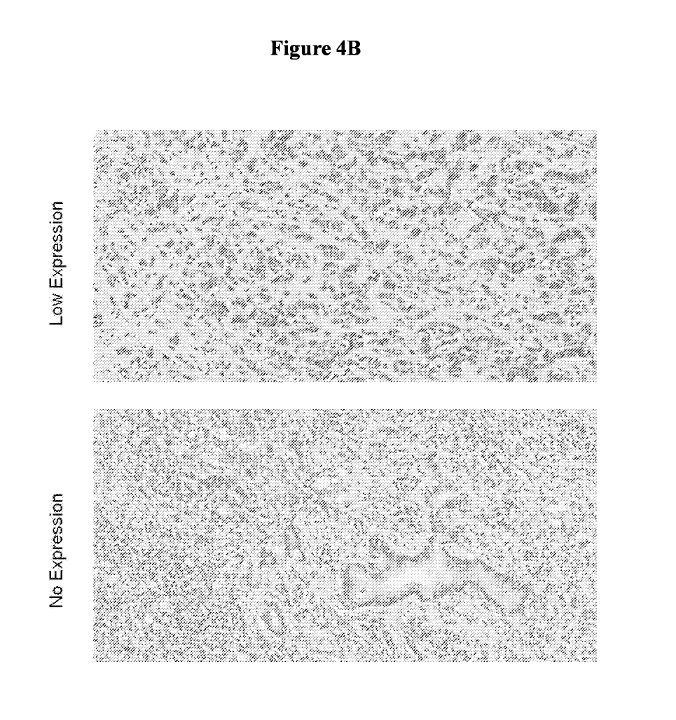

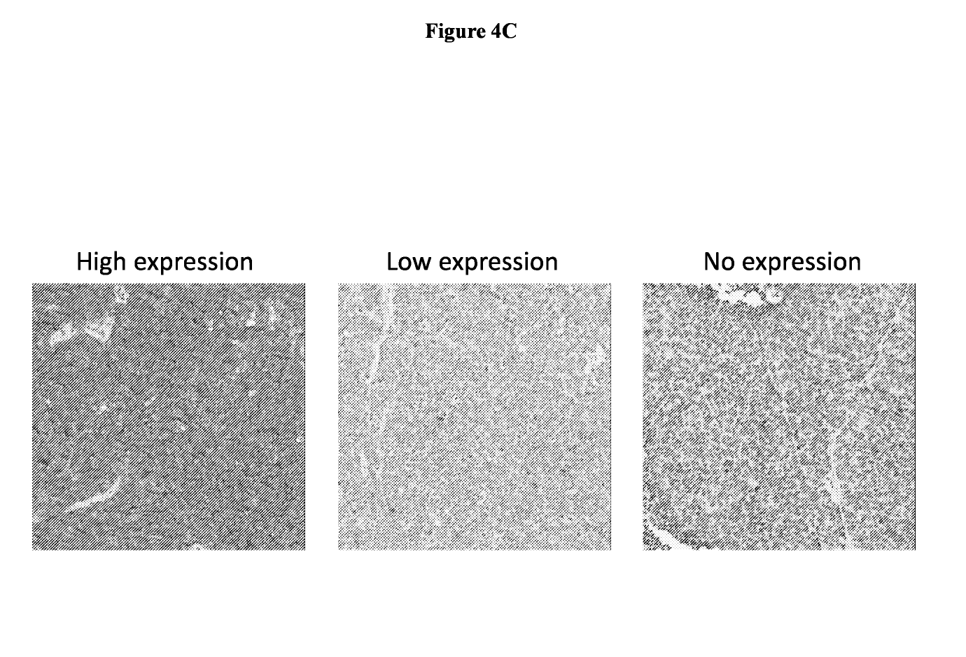

The slides were analyzed using an Aperio instrument (Leica Biosystems). The staining intensity of each tumor cell (0: no expression, 1: weak expression, 2: moderate expression, 3: strong expression) was measured and cells of each staining level were counted and a percentage for each type was calculated. The data was combined into a weighted H-score for each tissue section: H-score=[3×(% 3+cells)]+[2×(% 2+cells)]+[1×(% 1+cells)]. This calculation allows for a tope H-score of 300. Positive and negative controls included human tissue sections purchased from US Biomax (multiple normal tissue microarray) as well as patient-derived xenograft samples from the OncoMed tumor bank with known mRNA expression levels for NOTCH3 expression. Representative IHC results from pancreatic tumor tissue are shown in FIGS. 4A and 4B. Representative IHC results from lung tumor tissue are shown in FIG. 4C.

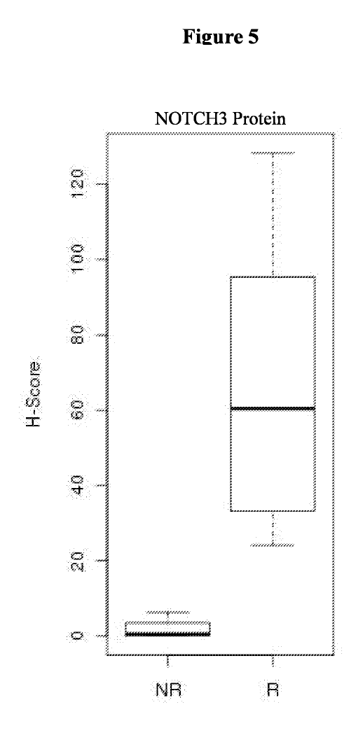

Using this IHC assay, xenograft pancreatic tumor samples from Example 1 were examined. As shown in FIG. 5, the IHC assay results showed good correlation with the identified responder and non-responder pancreatic tumors in the xenograft models. The distribution of NOTCH3 expression levels in non-responsive pancreatic tumors showed a clear separation from the distribution of NOTCH3 expression levels in responsive pancreatic tumors, paralleling the NOTCH3 gene expression results. These results provide a very strong basis for an IHC-based predictive assay for identifying pancreatic tumors likely to respond to treatment with the anti-NOTCH2/3 therapeutic antibody 59R5.

It is understood that the examples and embodiments described herein are for illustrative purposes only and that various modifications or changes in light thereof will be suggested to person skilled in the art and are to be included within the spirit and purview of this application.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

The sequences disclosed in the application are:

59R1/59R5 Heavy chain CDR1
(SEQ ID NO: 1)
SSSGMS
59R1/59R5 Heavy chain CDR2
(SEQ ID NO: 2)
VIASSGSNTYYADSVKG
59R1 Heavy chain CDR3
(SEQ ID NO: 3)
GIFFAI
59R5 Heavy chain CDR3
(SEQ ID NO: 4)
SIFYTT
Heavy chain CDR3 consensus sequence
(SEQ ID NO: 5)
(G/S)(I/S)F(F/Y)(T/A/P)(I/T/S/N)
Heavy chain CDR3 variant
(SEQ ID NO: 6)
SIFYPT
Heavy chain CDR3 variant
(SEQ ID NO: 7)
SSFFAS
Heavy chain CDR3 variant
(SEQ ID NO: 8)
SSFYAS
Heavy chain CDR3 variant
(SEQ ID NO: 9)
SSFFAT
Heavy chain CDR3 variant
(SEQ ID NO: 10)
SIFYPS
Heavy chain CDR3 variant
(SEQ ID NO: 11)
SSFFAN
59R1/59R5 Light chain CDR1
(SEQ ID NO: 12)
RASQSVRSNYLA
59R1/59R5 Light chain CDR2
(SEQ ID NO: 13)
GASSRAT
59R1/59R5 Light chain CDR3
(SEQ ID NO: 14)
QQYSNFPI
59R1 Heavy chain variable region with predicted signal
sequence underlined
(SEQ ID NO: 15)
MKHLWFFLLLVAAPRWVLSQVQLVESGGGLVQPGGSLRLSCAASGFTFSSSGMSWVRQAP
GKGLEWVSVIASSGSNTYYADSVKGRFTISRDNSKNTLYLQMNSLRAEDTAVYYCARGIF
FAIWGQGTLVTVSSA
59R5 Heavy chain variable region with predicted signal
sequence underlined
(SEQ ID NO: 16)
MKHLWFFLLLVAAPRWVLSEVQLVESGGGLVQPGGSLRLSCAASGFTFSSSGMSWVRQAP
GKGLEWVSVIASSGSNTYYADSVKGRFTISRDNSKNTLYLQMNSLRAEDTAVYYCARSIF
YTTWGQGTLVTVSSAST
59R1 Heavy chain variable region
(SEQ ID NO: 17)
QVQLVESGGGLVQPGGSLRLSCAASGFTFSSSGMSWVRQAPGKGLEWVSVIASSGSNTYY
ADSVKGRFTISRDNSKNTLYLQMNSLRAEDTAVYYCARGIFFAIWGQGTLVTVSSAST
59R5 Heavy chain variable region
(SEQ ID NO: 18)
EVQLVESGGGLVQPGGSLRLSCAASGFTFSSSGMSWVRQAPGKGLEWVSVIASSGSNTYY
ADSVKGRFTISRDNSKNTLYLQMNSLRAEDTAVYYCARSIFYTTWGQGTLVTVSSA
Variant 59R1 Heavy chain variable region
(SEQ ID NO: 19)
QVQLVESGGGLVQPGGSLRLSCAASGFTFSSSGMSWVRQAPGKGLEWVSVIASSGSNTYY
ADSVKGRFTISRDNSKNTLYLQMNSLRAEDTAVYYCARSIFYPTWGQGTLVTVSSA
Variant 59R1 Heavy chain variable region
(SEQ ID NO: 20)
QVQLVESGGGLVQPGGSLRLSCAASGFTFSSSGMSWVRQAPGKGLEWVSVIASSGSNTYY
ADSVKGRFTISRDNSKNTLYLQMNSLRAEDTAVYYCARSSFFASWGQGTLVTVSSA
Variant 59R1 Heavy chain variable region
(SEQ ID NO: 21)
QVQLVESGGGLVQPGGSLRLSCAASGFTFSSSGMSWVRQAPGKGLEWVSVIASSGSNTYY
ADSVKGRFTISRDNSKNTLYLQMNSLRAEDTAVYYCARSSFYASWGQGTLVTVSSA
Variant 59R1 Heavy chain variable region
(SEQ ID NO: 22)
QVQLVESGGGLVQPGGSLRLSCAASGFTFSSSGMSWVRQAPGKGLEWVSVIASSGSNTYY
ADSVKGRFTISRDNSKNTLYLQMNSLRAEDTAVYYCARSSFFATWGQGTLVTVSSA
Variant 59R1 Heavy chain variable region
(SEQ ID NO: 23)
QVQLVESGGGLVQPGGSLRLSCAASGFTFSSSGMSWVRQAPGKGLEWVSVIASSGSNTYY
ADSVKGRFTISRDNSKNTLYLQMNSLRAEDTAVYYCARSIFYPSWGQGTLVTVSSA
Variant 59R1 Heavy chain variable region
(SEQ ID NO: 24)
QVQLVESGGGLVQPGGSLRLSCAASGFTFSSSGMSWVRQAPGKGLEWVSVIASSGSNTYY
ADSVKGRFTISRDNSKNTLYLQMNSLRAEDTAVYYCARSSFFANWGQGTLVTVSSA
59R1/59R5 Light chain variable region with predicted signal
sequence underlined
(SEQ ID NO: 25)
MVLQTQVFISLLLWISGAYGDIVLTQSPATLSLSPGERATLSCRASQSVRSNYLAWYQQK
PGQAPRLLIYGASSRATGVPARFSGSGSGTDFTLTISSLEPEDFAVYYCQQYSNFPITFG
QGTKVEIKR
59R1/59R5 Light chain variable region
(SEQ ID NO: 26)
DIVLTQSPATLSLSPGERATLSCRASQSVRSNYLAWYQQKPGQAPRLLIYGASSRATGVP
ARFSGSGSGTDFTLTISSLEPEDFAVYYCQQYSNFPITFGQGTKVEIKR
59R5 Heavy chain with predicted signal sequence underlined
(SEQ ID NO: 27)
MKHLWFFLLLVAAPRWVLSEVQLVESGGGLVQPGGSLRLSCAASGFTFSSSGMSWVRQAP
GKGLEWVSVIASSGSNTYYADSVKGRFTISRDNSKNTLYLQMNSLRAEDTAVYYCARSIF
YTTWGQGTLVTVSSASTKGPSVFPLAPCSRSTSESTAALGCLVKDYFPEPVTVSWNSGAL
TSGVHTFPAVLQSSGLYSLSSVVTVPSSNFGTQTYTCNVDHKPSNTKVDKTVERKCCVEC
PPCPAPPVAGPSVFLFPPKPKDTLMISRTPEVTCVVVDVSHEDPEVQFNWYVDGVEVHNA
KTKPREEQFNSTFRVVSVLTVVHQDWLNGKEYKCKVSNKGLPAPIEKTISKTKGQPREPQ
VYTLPPSREEMTKNQVSLTCLVKGFYPSDIAVEWESNGQPENNYKTTPPMLDSDGSFFLY
SKLTVDKSRWQQGNVFSCSVMHEALHNHYTQKSLSLSPGK
59R5 Heavy chain without predicted signal sequence
(SEQ ID NO: 28)
EVQLVESGGGLVQPGGSLRLSCAASGFTFSSSGMSWVRQAPGKGLEWVSVIASSGSNTYY
ADSVKGRFTISRDNSKNTLYLQMNSLRAEDTAVYYCARSIFYTTWGQGTLVTVSSASTKG
PSVFPLAPCSRSTSESTAALGCLVKDYFPEPVTVSWNSGALTSGVHTFPAVLQSSGLYSL
SSVVTVPSSNFGTQTYTCNVDHKPSNTKVDKTVERKCCVECPPCPAPPVAGPSVFLFPPK
PKDTLMISRTPEVTCVVVDVSHEDPEVQFNWYVDGVEVHNAKTKPREEQFNSTFRVVSVL
TVVHQDWLNGKEYKCKVSNKGLPAPIEKTISKTKGQPREPQVYTLPPSREEMTKNQVSLT
CLVKGFYPSDIAVEWESNGQPENNYKTTPPMLDSDGSFFLYSKLTVDKSRWQQGNVFSCS
VMHEALHNHYTQKSLSLSPGK
59R1/59R5 Light chain with predicted signal sequence
underlined
(SEQ ID NO: 29)
MVLQTQVFISLLLWISGAYGDIVLTQSPATLSLSPGERATLSCRASQSVRSNYLAWYQQK
PGQAPRLLIYGASSRATGVPARFSGSGSGTDFTLTISSLEPEDFAVYYCQQYSNFPITFG
QGTKVEIKRTVAAPSVFIFPPSDEQLKSGTASVVCLLNNFYPREAKVQWKVDNALQSGNS
QESVTEQDSKDSTYSLSSTLTLSKADYEKHKVYACEVTHQGLSSPVTKSFNRGEC
59R1/59R5 Light chain without predicted signal sequence
(SEQ ID NO: 30)
DIVLTQSPATLSLSPGERATLSCRASQSVRSNYLAWYQQKPGQAPRLLIYGASSRATGVP
ARFSGSGSGTDFTLTISSLEPEDFAVYYCQQYSNFPITFGQGTKVEIKRTVAAPSVFIFP
PSDEQLKSGTASVVCLLNNFYPREAKVQWKVDNALQSGNSQESVTEQDSKDSTYSLSSTL
TLSKADYEKHKVYACEVTHQGLSSPVTKSFNRGEC
122M23 Sequences
122M23 Heavy chain variable region with signal sequence
(SEQ ID NO: 31)
ATGAAACACCTTTGGTTCTTCCTCCTCCTTGTCGCCGCTCCTAGATGGGTCCTCTCCCAA
GTCCAATTGCAGCAGTCCGGAGCCGAACTCGCACGGCCTGGCGCGAGCGTGAAGATGAGC
TGCAAGGCCTCGGGGTACACCTTTACCAAGTACACTATCCATTGGGTCGCCCAAAAGCCA
GGCCAGGGACTCGAGTGGATCGGGTACATTAACCCGTCGAATGATTACACCAACTATAAC
CAGACCTTCAAGGATAAAGCGACCCTGACGGCGGACAAGTCCTCAACCACTGCCTACATG
CAACTGATCTCGTTGACTTCGGCCGACAGCACCGTGTTCTACTGTGCTCGCGGTACTACG
CCGTACTCACTTGACTACTGGGGACAGGGAACCTCCGTGACTGTGAGCTCA
122M23 Heavy chain variable region without signal sequence
(SEQ ID NO: 32)
CAAGTCCAATTGCAGCAGTCCGGAGCCGAACTCGCACGGCCTGGCGCGAGCGTGAAGATG
AGCTGCAAGGCCTCGGGGTACACCTTTACCAAGTACACTATCCATTGGGTCGCCCAAAAG
CCAGGCCAGGGACTCGAGTGGATCGGGTACATTAACCCGTCGAATGATTACACCAACTAT
AACCAGACCTTCAAGGATAAAGCGACCCTGACGGCGGACAAGTCCTCAACCACTGCCTAC
ATGCAACTGATCTCGTTGACTTCGGCCGACAGCACCGTGTTCTACTGTGCTCGCGGTACT
ACGCCGTACTCACTTGACTACTGGGGACAGGGAACCTCCGTGACTGTGAGCTCA
122M23 Heavy chain variable region with predicted signal
sequence underlined
(SEQ ID NO: 33)
MKHLWFFLLLVAAPRWVLSQVQLQQSGAELARPGASVKMSCKASGYTFTKYTIHWVAQKP
GQGLEWIGYINPSNDYTNYNQTFKDKATLTADKSSTTAYMQLISLTSADSTVFYCARGTT
PYSLDYWGQGTSVTVSS
122M23 Heavy chain variable region without predicted signal
sequence
(SEQ ID NO: 34)
QVQLQQSGAELARPGASVKMSCKASGYTFTKYTIHWVAQKPGQGLEWIGYINPSNDYTNY
NQTFKDKATLTADKSSTTAYMQLISLTSADSTVFYCARGTTPYSLDYWGQGTSVTVSS
122M23 Light chain variable region with signal sequence
(SEQ ID NO: 35)
ATGAAACACCTGTGGTTCTTCCTCCTGCTGGTGGCAGCTCCCAGATGGGTCCTGTCCGAT
ATCGTGCTGACTCAGAGCCCAGCCTCGCTGGCCGTGTCGTTGGGTCAGCGGGCGACTATC
TCGTGCAGGGCCTCAGAATCAGTGGACAACTACGGGATGAGCTTCATGAACTGGTTTCAA
CAGAAGCCGGGTCAAAGCCCCAAATTGCTTATCTACGCTGCCAGCAACCAAGGATCCGGC
GTCCCGGCACGCTTTTCGGGAAGCGGCTCGGGCACCAATTTCTCCCTGAATATCCACCCT
ATGGAAGAAGATGATACCGCCGTGTACTTCTGCCTCCAGTCGAAGGAGGTGCCGTACACG
TTCGGAGGAGGAACTAAGCTGGAGATTAAGCGT
122M23 Light chain variable region without signal sequence
(SEQ ID NO: 36)
GATATCGTGCTGACTCAGAGCCCAGCCTCGCTGGCCGTGTCGTTGGGTCAGCGGGCGACT
ATCTCGTGCAGGGCCTCAGAATCAGTGGACAACTACGGGATGAGCTTCATGAACTGGTTT
CAACAGAAGCCGGGTCAAAGCCCCAAATTGCTTATCTACGCTGCCAGCAACCAAGGATCC
GGCGTCCCGGCACGCTTTTCGGGAAGCGGCTCGGGCACCAATTTCTCCCTGAATATCCAC
CCTATGGAAGAAGATGATACCGCCGTGTACTTCTGCCTCCAGTCGAAGGAGGTGCCGTAC
ACGTTCGGAGGAGGAACTAAGCTGGAGATTAAGCGT
122M23 Light chain variable region with predicted signal
sequence underlined
(SEQ ID NO: 37)
MKHLWFFLLLVAAPRWVLSDIVLTQSPASLAVSLGQRATISCRASESVDNYGMSFMNWFQ
QKPGQSPKLLIYAASNQGSGVPARFSGSGSGTNFSLNIHPMEEDDTAVYFCLQSKEVPYT
FGGGTKLEIKR
122M23 Light chain variable region without predicted signal
sequence
(SEQ ID NO: 38)
DIVLTQSPASLAVSLGQRATISCRASESVDNYGMSFMNWFQQKPGQSPKLLIYAASNQGS
GVPARFSGSGSGTNFSLNIHPMEEDDTAVYFCLQSKEVPYTFGGGTKLEIKR
122M23 Heavy chain CDR1
(SEQ ID NO: 39)
TKYTIH
122M23 Heavy chain CDR2
(SEQ ID NO: 40)
YINPSNDYTNYNQTFKD
122M23 Heavy chain CDR3
(SEQ ID NO: 41)
ARGTTPYSLDY
122M23 Light chain CDR1
(SEQ ID NO: 42)
RASESVDNYGMSFMN
122M23 Light chain CDR2
(SEQ ID NO: 43)
AASNQGS
122M23 Heavy chain CDR3
(SEQ ID NO: 44)
LQSKEVP
Human NOTCH3
(SEQ ID NO: 45)
MGPGARGRRRRRRPMSPPPPPPPVRALPLLLLLAGPGAAAPPCLDGSPCANGGRCTQLPS
REAACLCPPGWVGERCQLEDPCHSGPCAGRGVCQSSVVAGTARFSCRCPRGFRGPDCSLP
DPCLSSPCAHGARCSVGPDGRFLCSCPPGYQGRSCRSDVDECRVGEPCRHGGTCLNTPGS
FRCQCPAGYTGPLCENPAVPCAPSPCRNGGTCRQSGDLTYDCACLPGFEGQNCEVNVDDC
PGHRCLNGGTCVDGVNTYNCQCPPEWTGQFCTEDVDECQLQPNACHNGGTCFNTLGGHSC
VCVNGWTGESCSQNIDDCATAVCFHGATCHDRVASFYCACPMGKTGLLCHLDDACVSNPC
HEDAICDTNPVNGRAICTCPPGFTGGACDQDVDECSIGANPCEHLGRCVNTQGSFLCQCG
RGYTGPRCETDVNECLSGPCRNQATCLDRIGQFTCICMAGFTGTYCEVDIDECQSSPCVN
GGVCKDRVNGFSCTCPSGFSGSTCQLDVDECASTPCRNGAKCVDQPDGYECRCAEGFEGT
LCDRNVDDCSPDPCHHGRCVDGIASFSCACAPGYTGTRCESQVDECRSQPCRHGGKCLDL
VDKYLCRCPSGTTGVNCEVNIDDCASNPCTFGVCRDGINRYDCVCQPGFTGPLCNVEINE
CASSPCGEGGSCVDGENGFRCLCPPGSLPPLCLPPSHPCAHEPCSHGICYDAPGGFRCVC
EPGWSGPRCSQSLARDACESQPCRAGGTCSSDGMGFHCTCPPGVQGRQCELLSPCTPNPC
EHGGRCESAPGQLPVCSCPQGWQGPRCQQDVDECAGPAPCGPHGICTNLAGSFSCTCHGG
YTGPSCDQDINDCDPNPCLNGGSCQDGVGSFSCSCLPGFAGPRCARDVDECLSNPCGPGT
CTDHVASFTCTCPPGYGGFHCEQDLPDCSPSSCFNGGTCVDGVNSFSCLCRPGYTGAHCQ
HEADPCLSRPCLHGGVCSAAHPGFRCTCLESFTGPQCQTLVDWCSRQPCQNGGRCVQTGA
YCLCPPGWSGRLCDIRSLPCREAAAQIGVRLEQLCQAGGQCVDEDSSHYCVCPEGRTGSH
CEQEVDPCLAQPCQHGGTCRGYMGGYMCECLPGYNGDNCEDDVDECASQPCQHGGSCIDL
VARYLCSCPPGTLGVLCEINEDDCGPGPPLDSGPRCLHNGTCVDLVGGFRCTCPPGYTGL
RCEADINECRSGACHAAHTRDCLQDPGGGFRCLCHAGFSGPRCQTVLSPCESQPCQHGGQ
CRPSPGPGGGLTFTCHCAQPFWGPRCERVARSCRELQCPVGVPCQQTPRGPRCACPPGLS
GPSCRSFPGSPPGASNASCAAAPCLHGGSCRPAPLAPFFRCACAQGWTGPRCEAPAAAPE
VSEEPRCPRAACQAKRGDQRCDRECNSPGCGWDGGDCSLSVGDPWRQCEALQCWRLFNNS
RCDPACSSPACLYDNFDCHAGGRERTCNPVYEKYCADHFADGRCDQGCNTEECGWDGLDC
ASEVPALLARGVLVLTVLLPPEELLRSSADFLQRLSAILRTSLRFRLDAHGQAMVFPYHR
PSPGSEPRARRELAPEVIGSVVMLEIDNRLCLQSPENDHCFPDAQSAADYLGALSAVERL
DFPYPLRDVRGEPLEPPEPSVPLLPLLVAGAVLLLVILVLGVMVARRKREHSTLWFPEGF
SLHKDVASGHKGRREPVGQDALGMKNMAKGESLMGEVATDWMDTECPEAKRLKVEEPGMG
AEEAVDCRQWTQHHLVAADIRVAPAMALTPPQGDADADGMDVNVRGPDGFTPLMLASFCG
GALEPMPTEEDEADDTSASIISDLICQGAQLGARTDRTGETALHLAARYARADAAKRLLD
AGADTNAQDHSGRTPLHTAVTADAQGVFQILIRNRSTDLDARMADGSTALILAARLAVEG
MVEELIASHADVNAVDELGKSALHWAAAVNNVEATLALLKNGANKDMQDSKEETPLFLAA
REGSYEAAKLLLDHFANREITDHLDRLPRDVAQERLHQDIVRLLDQPSGPRSPPGPHGLG
PLLCPPGAFLPGLKAAQSGSKKSRRPPGKAGLGPQGPRGRGKKLTLACPGPLADSSVTLS
PVDSLDSPRPFGGPPASPGGFPLEGPYAAATATAVSLAQLGGPGRAGLGRQPPGGCVLSL
GLLNPVAVPLDWARLPPPAPPGPSFLLPLAPGPQLLNPGTPVSPQERPPPYLAVPGHGEE
YPAAGAHSSPPKARFLRVPSEHPYLTPSPESPEHWASPSPPSLSDWSESTPSPATATGAM
ATTTGALPAQPLPLSVPSSLAQAQTQLGPQPEVTPKRQVLA
FLAG tag
(SEQ ID NO: 46)
DYKDDDDK

What we claim is:

1. An isolated antibody that specifically binds the extracellular domain of human NOTCH3, which comprises:
(a) a heavy chain CDR1 comprising TKYTIH (SEQ ID NO:39), a heavy chain CDR2 comprising YINPSNDYTNYNQTFKD (SEQ ID NO:40), and a heavy chain CDR3 comprising ARGTTPYSLDY (SEQ ID NO:41); and
(b) a light chain CDR1 comprising RASESVDNYGMSFMN (SEQ ID NO:42), a light chain CDR2 comprising AASNQGS (SEQ ID NO:43), and a light chain CDR3 comprising LQSKEVP (SEQ ID NO:44).

2. The antibody of claim 1, which binds within EGF repeats 9-14 of NOTCH3.

3. The antibody of claim 1, which binds within amino acids 350-580 of NOTCH3 (SEQ ID NO:45).

4. The antibody of any one of claims 1-3, which comprises:
(a) a heavy chain variable region having at least 90% sequence identity to SEQ ID NO:34; and
(b) a light chain variable region having at least 90% sequence identity to SEQ ID NO:38.

5. The antibody of any one of claims 1-3, which comprises:
(a) a heavy chain variable region having at least 95% sequence identity to SEQ ID NO:34; and
(b) a light chain variable region having at least 95% sequence identity to SEQ ID NO:38.

6. The antibody of any one of claims 1-3, which comprises a heavy chain variable region comprising SEQ ID NO:34 and a light chain variable region comprising SEQ ID NO:38.

7. The antibody of any one of claims 1-6, which is a recombinant antibody, a monoclonal antibody, a chimeric antibody, a bispecific antibody, a humanized antibody, a human antibody, an IgG1 antibody, an IgG2 antibody, or an antibody fragment comprising an antigen binding site.

8. An isolated antibody comprising a heavy chain variable region encoded by the plasmid deposited with ATCC as PTA-121156 and a light chain variable region encoded by the plasmid deposited with ATCC as PTA-121155.

9. An antibody comprising a heavy chain encoded by the plasmid deposited with ATCC as PTA-121156 and a light chain encoded by the plasmid deposited with ATCC as PTA-121155.

10. An antibody produced by a hybridoma deposited with ATCC as PTA-121154.

11. An antibody that competes with the antibody of any one of claims 1-10 for binding to human NOTCH3.

12. The antibody of any one of claims 1-11, which is detectably labeled.

13. The antibody of claim 12, wherein the detectable label is selected from the group consisting of: an affinity label, an enzymatic label, a fluorescent label, a radioisotope label, and a magnetic label.

14. An isolated polynucleotide comprising a polynucleotide that encodes the antibody of any one of claims 1-11.

15. An isolated polynucleotide comprising SEQ ID NO:31, SEQ ID NO:32, SEQ ID NO:35, or SEQ ID NO:36.

16. A vector comprising the polynucleotide of claim 14 or claim 15.

17. A cell producing or comprising the antibody of any one of claims 1-11.

18. A hybridoma producing the antibody of any one of claims 1-11.

19. A cell comprising the polynucleotide of claim 14 or claim 15.

20. A cell comprising the vector of claim 16.

21. A method of detecting the extracellular domain of human NOTCH3 in a pancreatic tumor sample, comprising:
(a) contacting the sample with an antibody of any one of claims 1-11; and
(b) determining if the antibody binds the sample.

22. The method of claim 21, wherein the sample is a biopsy sample.

23. The method of claim 21 or claim 22, wherein the sample is a formalin-fixed paraffin embedded (FFPE) sample.

24. The method of any one of claims 21-23, further comprising:
(c) determining the level of NOTCH3 expression in the sample.

25. The method of claim 24, further comprising:
(d) comparing the level of NOTCH3 expression in the sample to a pre-determined level of NOTCH3 expression.

26. The method of claim 25, wherein the pre-determined level of NOTCH3 expression is a level of NOTCH3 expression in a reference tumor sample, a reference normal tissue sample, a series of reference tumor samples, or a series of reference normal tissue samples.

27. The method of any one of claims 21-26, wherein the method is an immunohistochemistry (IHC) assay.

28. The method of any one of claims 24-27, wherein the determining the level of NOTCH3 expression in the sample comprises an H-score evaluation.

29. A method of identifying a pancreatic tumor likely to be responsive to a first antibody that specifically binds human NOTCH2/3, the method comprising determining the level of NOTCH3 expression in a sample obtained from the pancreatic tumor, wherein the level of NOTCH3 expression is determined using a second antibody that specifically binds the extracellular domain of human NOTCH3, and the second antibody comprises a heavy chain CDR1 comprising TKYTIH (SEQ ID NO:39), a heavy chain CDR2 comprising YINPSNDYTNYNQTFKD (SEQ ID NO:40), a heavy chain CDR3 comprising ARGTTPYSLDY (SEQ ID NO:41); a light chain CDR1 comprising RASESVDNYGMSFMN (SEQ ID NO:42), a light chain CDR2 comprising AASNQGS (SEQ ID NO:43), and a light chain CDR3 comprising LQSKEVP (SEQ ID NO:44).

30. A method of identifying a subject with a pancreatic tumor as likely to be responsive to a first antibody that specifically binds human NOTCH2/3, the method comprising:
(a) determining the level of NOTCH3 expression in a sample obtained from the pancreatic tumor, wherein the level of NOTCH3 expression is determined using a second antibody that specifically binds the extracellular domain of human NOTCH3, and the second antibody comprises a heavy chain CDR1 comprising TKYTIH (SEQ ID NO:39), a heavy chain CDR2 comprising YINPSNDYTNYNQTFKD (SEQ ID NO:40), a heavy chain CDR3 comprising ARGTTPYSLDY (SEQ ID NO:41); a light chain CDR1 comprising RASESVDNYGMSFMN (SEQ ID NO:42), a light chain CDR2 comprising AASNQGS (SEQ ID NO:43), and a light chain CDR3 comprising LQSKEVP (SEQ ID NO:44); and
(b) identifying the subject as likely to be responsive or non-responsive to treatment with the first antibody that specifically binds human NOTCH2/3 based upon the level of NOTCH3 expression in the pancreatic tumor.

31. A method for selecting a subject with a pancreatic tumor for treatment with a first antibody that specifically binds human NOTCH2/3, the method comprising:
(a) determining the level of NOTCH3 expression in a sample obtained from the pancreatic tumor, wherein the level of NOTCH3 expression is determined using a second antibody that specifically binds the extracellular domain of human NOTCH3, and the second antibody comprises a heavy chain CDR1 comprising TKYTIH (SEQ ID NO:39), a heavy chain CDR2 comprising YINPSNDYTNYNQTFKD (SEQ ID NO:40), a heavy chain CDR3 comprising ARGTTPYSLDY (SEQ ID NO:41); a light chain CDR1 comprising RASESVDNYGMSFMN (SEQ ID NO:42), a light chain CDR2 comprising AASNQGS (SEQ ID NO:43), and a light chain CDR3 comprising LQSKEVP (SEQ ID NO:44);
(b) identifying the pancreatic tumor as likely to be responsive or non-responsive to treatment with the first antibody that specifically binds human NOTCH2/3 based upon the level of NOTCH3 expression in the pancreatic tumor; and
(c) selecting the subject for treatment with the first antibody if the pancreatic tumor is identified as likely to be responsive to treatment.

32. A method for stratifying a pancreatic cancer patient population for treatment with a first antibody that specifically binds human NOTCH2/3, the method comprising:
(a) determining the level of NOTCH3 expression samples from the patients, wherein the level of NOTCH3 expression is determined using a second antibody that specifically binds the extracellular domain of human NOTCH3, and the second antibody comprises a heavy chain CDR1 comprising TKYTIH (SEQ ID NO:39), a heavy chain CDR2 comprising YINPSNDYTNYNQTFKD (SEQ ID NO:40), a heavy chain CDR3 comprising ARGTTPYSLDY (SEQ ID NO:41); a light chain CDR1 comprising RASESVDNYGMSFMN (SEQ ID NO:42), a light chain CDR2 comprising AASNQGS (SEQ ID NO:43), and a light chain CDR3 comprising LQSKEVP (SEQ ID NO:44);
(b) stratifying the patient population based on the expression level of NOTCH3 in the samples.

33. The method of any one of claims 29-32, wherein the sample is a biopsy sample.

34. The method of any one of claims 29-33, wherein the sample is a formalin-fixed paraffin embedded (FFPE) sample.

35. The method of any one of claims 29-35, wherein the level of NOTCH3 expression in the sample is compared to a pre-determined level of NOTCH3 expression.

36. The method of claim 35, wherein the pre-determined level of NOTCH3 expression is a level of NOTCH3 expression in a reference tumor sample, a reference normal tissue sample, a series of reference tumor samples, or a series of reference normal tissue samples.

37. The method of any one of claims 29-36, wherein the level of NOTCH3 expression is determined using an immunohistochemistry (IHC) assay.

38. The method of any one of claims 29-37, wherein the level of NOTCH3 expression is determined using an assay which comprises an H-score evaluation.

39. The method of any one of claims 29-38, wherein the first antibody that specifically binds human NOTCH2/3 comprises:
(a) a heavy chain CDR1 comprising SSSGMS (SEQ ID NO:1), a heavy chain CDR2 comprising VIASSGSNTYYADSVKG (SEQ ID NO:2), and a heavy chain CDR3 comprising SIFYTT (SEQ ID NO:4); and
(b) a light chain CDR1 comprising RASQSVRSNYLA (SEQ ID NO:12), a light chain CDR2 comprising GASSRAT (SEQ ID NO:13), and a light chain CDR3 comprising QQYSNFPI (SEQ ID NO:14).

40. The method of claim 39, wherein the first antibody that specifically binds human NOTCH2/3 comprises:
(a) a heavy chain variable region having at least 90% sequence identity to SEQ ID NO:18; and
(b) a light chain variable region having at least 90% sequence identity to SEQ ID NO:26.

41. The method of claim 39, wherein the first antibody that specifically binds human NOTCH2/3 comprises:
(a) a heavy chain variable region having at least 95% sequence identity to SEQ ID NO:18; and
(b) a light chain variable region having at least 95% sequence identity to SEQ ID NO:26.

42. The method of claim 39, wherein the first antibody that specifically binds human NOTCH2/3 comprises:
(a) a heavy chain variable region comprising SEQ ID NO:18; and
(b) a light chain variable region comprising SEQ ID NO:26.

43. The method of claim 39, wherein the first antibody that specifically binds human NOTCH2/3 comprises the same heavy chain variable region and the same light chain variable region as an antibody encoded by the plasmid deposited with ATCC as PTA-9547.

44. The method of claim 39, wherein the first antibody that specifically binds human NOTCH2/3 is encoded by the plasmid deposited with ATCC as PTA-9547.

45. The method of any one of claims 39-44, wherein first antibody that specifically binds human NOTCH2/3 is a recombinant antibody, a monoclonal antibody, a chimeric antibody, a humanized antibody, a human antibody, a bispecific antibody, or an antibody fragment.

46. A kit comprising the antibody of any one of claims 1-11 and instructions for use.

47. A diagnostic composition comprising the antibody of any one of claims 1-11.
